(19) United States
(12) Patent Application Publication
(10) Pub. No.: US 2025/0376459 A1
(43) Pub. Date: Dec. 11, 2025

(54) CRYSTALLINE FORMS OF A TYK2 INHIBITOR AND USES THEREOF

(71) Applicant: Alumis Inc., South San Francisco, CA (US)

(72) Inventors: Kolbot By, South San Francisco, CA (US); Travis Remarchuk, South San Francisco, CA (US)

(21) Appl. No.: 19/110,181

(22) PCT Filed: Sep. 12, 2023
(86) PCT No.: PCT/US2023/073918

§ 371 Date: Mar. 10, 2025

Related U.S. Application Data
(60) Provisional application No. 63/405,582, filed on Sep. 12, 2022.

(51) Int. Cl. C07D 401/12; A61K 31/4439; C07C 309/04

(52) U.S. Cl. CPC C07D 401/12; A61K 31/4439; C07C 309/04; C07B 2200/13

(57) ABSTRACT

The disclosure is in part directed to crystalline salt forms of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropane carboxamide, pharmaceutical compositions thereof, and methods of use.

### CROSS REFERENCE TO RELATED APPLICATIONS

This application claims the benefit of, and priority to, U.S. Provisional Patent Application No. 63/405,582 filed Sep. 12, 2022; the content of which is hereby incorporated by reference herein in its entirety.

### BACKGROUND

TYK2 is a non-receptor tyrosine kinase member of the Janus kinase (JAKs) family of protein kinases. TYK2 activation has been linked to diseases and disorders such as, for example, autoimmune disorders, inflammatory disorders, proliferative disorders (e.g., cancer), endocrine disorders, and neurological disorders. For example, TYK2 activation has been linked to inflammatory bowel disease (IBD), Crohn's disease, rheumatoid arthritis, psoriasis, systemic lupus erythematosus, ulcerative colitis, psoriatic arthritis, and systemic sclerosis. TYK2 also plays a role in respiratory diseases such as asthma, chronic obstructive pulmonary disease (COPD), lung cancer, and cystic fibrosis. Accordingly, compounds that inhibit the activity of TYK2 are beneficial, especially those with selectivity over JAK2. Such compounds should deliver a pharmacological response that favorably treats one or more of the conditions described herein without the side-effects associated with the inhibition of JAK2.

Polymorphism is the ability of a substance to crystallize in more than one crystal lattice arrangement. Crystallization, or polymorphism, can influence many aspects of the solid-state properties of a drug substance. A crystalline substance may differ considerably from an amorphous form, and different crystal modifications of a substance may differ considerably from one another in many respects including solubility, dissolution rate and/or bioavailability. Generally, it is difficult to predict whether a given compound will form any crystalline solid-state forms. It is even more difficult to predict the physical properties of these crystalline solid-state forms. Therefore, it can be advantageous to have a crystalline form of a therapeutic agent for certain formulations and/or for manufacturing processes.

### SUMMARY

The present disclosure is directed, at least in part, to crystalline salt forms of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide.

For example, disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, methanesulfonic acid salt, characterized by a powder X-ray diffraction pattern having a characteristic peak in degrees 2θ at about 24.0, for example, characterized by a powder X-ray diffraction pattern having characteristic peaks in degrees 2θ at about 12.7, 17.1, and 24.0, for example, characterized by a powder X-ray diffraction pattern having characteristic peaks in degrees 2θ at about 9.5, 12.7, 17.1, 19.1, 20.5, and 24.0, for example, characterized by a powder X-ray diffraction pattern having characteristic peaks in degrees 2θ at about 9.5, 12.7, 15.5, 17.1, 19.1, 20.5, 24.0, 24.3, and 26.9, for example, characterized by a powder X-ray diffraction pattern having characteristic peaks in degrees 2θ at about 7.8, 9.5, 12.7, 15.5, 17.1, 19.1, 20.5, 21.0, 23.7, 24.0, 24.3, and 26.9, for example, characterized by a powder X-ray diffraction pattern having characteristic peaks in degrees 2θ at about 7.8, 9.5, 12.7, 15.5, 17.1, 19.1, 19.4, 20.5, 20.8, 21.0, 23.7, 24.0, 24.3, 25.5, and 26.9.

Also disclosed herein is a different crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, methanesulfonic acid salt, characterized by a powder X-ray diffraction pattern having a characteristic peak in degrees 2θ at about 9.9, for example, characterized by a powder X-ray diffraction pattern having characteristic peaks in degrees 2θ at about 7.5, 9.9, and 20.0, for example, characterized by a powder X-ray diffraction pattern having characteristic peaks in degrees 2θ at about 7.5, 9.9, 13.7, 18.2, 20.0, and 22.5, for example, characterized by a powder X-ray diffraction pattern having characteristic peaks in degrees 2θ at about 7.5, 9.2, 9.9, 13.7, 18.2, 20.0, 23.9, 22.5, and 26.5, for example, characterized by a powder X-ray diffraction pattern having characteristic peaks in degrees 2θ at about 7.5, 8.3, 9.2, 9.9, 10.4, 13.7, 18.2, 20.0, 21.1, 23.9, 22.5, and 26.5.

Further disclosed herein is a different crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, methanesulfonic acid salt, characterized by a powder X-ray diffraction pattern having a characteristic peak in degrees 2θ at about 21.0, for example, characterized by a powder X-ray diffraction pattern having characteristic peaks in degrees 2θ at about 9.0, 18.0, and 21.0, for example, characterized by a powder X-ray diffraction pattern having characteristic peaks in degrees 2θ at about 7.1, 8.1, 9.0, 18.0, 21.0, and 23.3, for example, characterized by a powder X-ray diffraction pattern having characteristic peaks in degrees 2θ at about 7.1, 8.1, 9.0, 10.4, 18.0, 20.6, 21.0, 23.3, and 28.5, for example, characterized by a powder X-ray diffraction pattern having characteristic peaks in degrees 2θ at about 7.1, 8.1, 9.0, 10.4, 16.5, 18.0, 20.6, 21.0, 23.3, 24.0, 24.2, and 28.5, for example, characterized by a powder X-ray diffraction pattern having characteristic peaks in degrees 2θ at about 7.1, 8.1, 9.0, 10.4, 11.1, 15.6, 16.5, 17.5, 18.0, 20.6, 21.0, 23.3, 24.0, 24.2, and 28.5.

Also disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, fumaric acid salt, characterized by a powder X-ray diffraction pattern having a characteristic peak in degrees 2θ at about 26.1, for example, characterized by a powder X-ray diffraction pattern having characteristic peaks in degrees 2θ at about 8.0, 23.3, and 26.1, for example, characterized by a powder X-ray diffraction pattern having characteristic peaks in degrees 2θ at about 8.0, 13.5, 22.3, 23.3, 25.6, and 26.1, for example, characterized by a powder X-ray diffraction pattern having characteristic peaks in degrees 2θ at about 4.2, 8.0, 8.3, 13.5, 21.0, 22.3, 23.3, 25.6, and 26.1, for example, characterized by a powder X-ray diffraction pattern having characteristic peaks in degrees 2θ at about 4.2, 8.0, 8.3, 10.7, 13.5, 18.5, 21.0, 21.3, 22.3, 23.3, 25.6, and 26.1, for example, characterized by a powder X-ray diffraction pattern having characteristic peaks in degrees 2θ at about 4.2, 8.0, 8.3, 10.7, 12.5, 13.5, 18.5, 21.0, 22.0, 21.3, 21.7, 22.3, 23.3, 25.6, and 26.1.

Disclosed herein, for example, is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, hydrogen bromide salt, characterized by a powder X-ray diffraction pattern having a characteristic peak in degrees 2θ at about 9.7, for example, characterized by a powder X-ray diffraction pattern having characteristic peaks in degrees 2θ at about 9.7, 19.5, and 20.5, for example, characterized by a powder X-ray diffraction pattern having characteristic peaks in degrees 2θ at about 9.7, 19.5, 20.5, 21.2, 23.3, and 26.4, for example, characterized by a powder X-ray diffraction pattern having characteristic peaks in degrees 2θ at about 9.7, 17.4, 19.5, 20.5, 21.2, 23.3, 26.4, 27.8, and 29.4, for example, characterized by a powder X-ray diffraction pattern having characteristic peaks in degrees 2θ at about 9.7, 17.1, 17.4, 19.5, 20.5, 20.9, 21.2, 22.9, 23.3, 26.4, 27.8, and 29.4, for example, characterized by a powder X-ray diffraction pattern having characteristic peaks in degrees 2θ at about 9.7, 17.1, 17.4, 19.5, 20.5, 20.9, 21.2, 22.9, 23.3, 24.8, 26.4, 27.8, 29.4, 30.8, and 32.2.

Further disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, phosphoric acid salt, characterized by a powder X-ray diffraction pattern having a characteristic peak in degrees 2θ at about 4.8, for example, characterized by a powder X-ray diffraction pattern having characteristic peaks in degrees 2θ at about 4.8, 6.2, and 9.6, for example, characterized by a powder X-ray diffraction pattern having characteristic peaks in degrees 2θ at about 4.8, 6.2, 7.9, 9.6, 12.6, and 22.2, for example, characterized by a powder X-ray diffraction pattern having characteristic peaks in degrees 2θ at about 4.8, 6.2, 7.9, 9.6, 12.6, 16.7, 18.5, 21.5, and 22.2.

In addition, disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, sulfuric acid salt, characterized by a powder X-ray diffraction pattern having a characteristic peak in degrees 2θ at about 21.2, for example, characterized by a powder X-ray diffraction pattern having characteristic peaks in degrees 2θ at about 8.4, 16.9, and 21.2, for example, characterized by a powder X-ray diffraction pattern having characteristic peaks in degrees 2θ at about 8.4, 16.9, 20.9, 21.2, 22.2, and 23.5, for example, characterized by a powder X-ray diffraction pattern having characteristic peaks in degrees 2θ at about 7.3, 8.4, 16.9, 18.0, 20.9, 21.2, 21.8, 22.2, and 23.5, for example, characterized by a powder X-ray diffraction pattern having characteristic peaks in degrees 2θ at about 7.3, 8.4, 9.0, 15.9, 16.9, 18.0, 20.9, 21.2, 21.8, 22.2, 23.1, and 23.5, for example, characterized by a powder X-ray diffraction pattern having characteristic peaks in degrees 2θ at about 7.3, 8.4, 9.0, 15.4, 15.9, 16.9, 18.0, 20.6, 20.9, 21.2, 21.8, 22.2, 23.1, 23.5, and 27.8.

Also disclosed herein is a different crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, sulfuric acid salt, characterized by a powder X-ray diffraction pattern having a characteristic peak in degrees 2θ at about 16.1, for example, characterized by a powder X-ray diffraction pattern having characteristic peaks in degrees 2θ at about 16.1, 19.2, and 23.9, for example, characterized by a powder X-ray diffraction pattern having characteristic peaks in degrees 2θ at about 9.6, 16.1, 19.2, 19.9, 21.6, and 23.9, for example, characterized by a powder X-ray diffraction pattern having characteristic peaks in degrees 2θ at about 9.6, 12.6, 16.1, 19.2, 19.9, 21.6, 23.9, 24.2, and 25.4, for example, characterized by a powder X-ray diffraction pattern having characteristic peaks in degrees 2θ at about 8.2, 9.6, 12.6, 16.1, 18.0, 19.2, 19.9, 21.6, 23.9, 24.2, 24.6, and 25.4, for example, characterized by a powder X-ray diffraction pattern having characteristic peaks in degrees 2θ at about 8.2, 9.6, 12.6, 15.4, 16.1, 18.0, 19.2, 19.9, 20.3, 21.6, 23.9, 24.2, 24.6, 25.4, and 28.2.

Further disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, p-toluenesulfonic acid salt, characterized by a powder X-ray diffraction pattern having a characteristic peak in degrees 2θ at about 9.8, for example, characterized by a powder X-ray diffraction pattern having characteristic peaks in degrees 2θ at about 9.8, 19.8, and 22.7, for example, characterized by a powder X-ray diffraction pattern having characteristic peaks in degrees 2θ at about 6.6, 7.2, 9.8, 19.8, 20.2, and 22.7, for example, characterized by a powder X-ray diffraction pattern having characteristic peaks in degrees 2θ at about 6.6, 7.2, 9.8, 16.1, 19.8, 20.2, 22.7, 24.9, and 25.4, for example, characterized by a powder X-ray diffraction pattern having characteristic peaks in degrees 2θ at about 6.6, 7.2, 9.8, 15.1, 15.8, 16.1, 19.5, 19.8, 20.2, 22.7, 24.9, and 25.4, for example, characterized by a powder X-ray diffraction pattern having characteristic peaks in degrees 2θ at about 6.6, 7.2, 9.8, 13.3, 15.1, 15.8, 16.1, 19.5, 19.8, 20.2, 21.4, 22.7, 24.9, 25.4, and 26.5.

Further disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide, bis-hydrochloride salt, characterized by a powder X-ray diffraction pattern having a characteristic peak in degrees 2θ at about 9.4, for example, characterized by a powder X-ray diffraction pattern having characteristic peaks in degrees 2θ at about 5.4, 6.2, and 9.4, for example, characterized by a powder X-ray diffraction pattern having characteristic peaks in degrees 2θ at about 5.4, 6.2, 9.4, 24.3, 26.2, and 26.7, for example, characterized by a powder X-ray diffraction pattern having characteristic peaks in degrees 2θ at about 5.4, 6.2, 9.4, 10.3, 24.3, 24.7, 26.2, 26.7, and 31.0, for example, characterized by a powder X-ray diffraction pattern having characteristic peaks in degrees 2θ at about 5.4, 6.2, 9.4, 10.3, 10.8, 16.0, 18.5, 24.3, 24.7, 26.2, 26.7, and 31.0.

N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide is, for example, a modulator of tyrosine kinase 2 (TYK2), e.g., an inhibitor of TYK2, and is represented by:

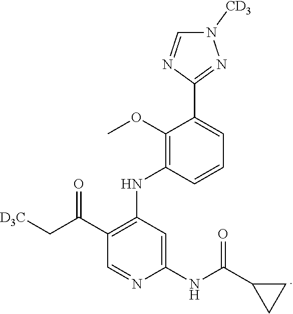 [SMILES: [2H]C([2H])([2H])CC(=O)c1cnc(NC(=O)C2CC2)cc1Nc1cccc(-c2ncn(C([2H])([2H])[2H])n2)c1OC]

Further contemplated herein is a pharmaceutical composition comprising a disclosed crystalline salt form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide and a pharmaceutically acceptable excipient, for example, a composition that is formulated for oral administration. Further contemplated herein is a drug substance comprising at least a detectable amount of a disclosed crystalline salt form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide. For example, disclosed herein is a drug substance comprising substantially pure crystalline salt form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide.

Also provided herein is a method of treating a TYK2-mediated disorder in a patient in need thereof, comprising administering to the patient an effective amount of a disclosed crystalline salt form of N-(4-((2-methoxy-3-(1-(methyl-d₃)-1H-1,2,4-triazol-3-yl)phenyl)amino)-5-(propanoyl-3,3,3-d₃)pyridin-2-yl)cyclopropanecarboxamide.

For example, provided herein is a method of treating one or more of. Crohn's disease, rheumatoid arthritis, psoriasis, systemic lupus erythematosus, ulcerative colitis, psoriatic arthritis, and systemic sclerosis in a patient in need thereof, comprising administering to the patient an effective amount of a disclosed crystalline salt form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide.

### BRIEF DESCRIPTION OF THE DRAWINGS

FIG. 1 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid (Form 1-1).

FIG. 2 depicts the differential scanning calorimetry (DSC) profile of Form 1-1.

FIG. 3 depicts the thermogravimetric analysis (TGA) profile of Form 1-1.

FIG. 4 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt (Form 1-2).

FIG. 5 depicts the differential scanning calorimetry (DSC) profile of Form 1-2.

FIG. 6 depicts the thermogravimetric analysis (TGA) profile of Form 1-2.

FIG. 7 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt (Form 1-3).

FIG. 8 depicts the differential scanning calorimetry (DSC) profile of Form 1-3.

FIG. 9 depicts the thermogravimetric analysis (TGA) profile of Form 1-3.

FIG. 10 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, fumaric acid salt (Form 2).

FIG. 11 depicts the differential scanning calorimetry (DSC) profile of Form 2.

FIG. 12 depicts the thermogravimetric analysis (TGA) profile of Form 2.

FIG. 13 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, hydrogen bromide salt (Form 3).

FIG. 14 depicts the differential scanning calorimetry (DSC) profile of Form 3.

FIG. 15 depicts the thermogravimetric analysis (TGA) profile of Form 3.

FIG. 16 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, phosphoric acid salt (Form 4).

FIG. 17 depicts the differential scanning calorimetry (DSC) profile of Form 4.

FIG. 18 depicts the thermogravimetric analysis (TGA) profile of Form 4.

FIG. 19 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt (Form 5-1).

FIG. 20 depicts the differential scanning calorimetry (DSC) profile of Form 5-1.

FIG. 21 depicts the thermogravimetric analysis (TGA) profile of Form 5-1.

FIG. 22 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt (Form 5-2).

FIG. 23 depicts the differential scanning calorimetry (DSC) profile of Form 5-2.

FIG. 24 depicts the thermogravimetric analysis (TGA) profile of Form 5-2.

FIG. 25 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, p-toluenesulfonic acid salt (Form 6).

FIG. 26 depicts the differential scanning calorimetry (DSC) profile of Form 6.

FIG. 27 depicts the thermogravimetric analysis (TGA) profile of Form 6.

FIG. 28 depicts an X-ray powder diffraction (XRPD) pattern of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, bis-hydrochloride salt (Form 7).

FIG. 29 depicts the differential scanning calorimetry (DSC) profile of Form 7.

FIG. 30 depicts the thermogravimetric analysis (TGA) profile of Form 7.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “crystalline form” refers to a crystal form or modification that can be characterized by analytical methods such as, e.g., X-ray powder diffraction (XRPD) and/or Differential scanning calorimetry (DSC). The crystalline compounds disclosed herein can exist in solvated as well as unsolvated forms with solvents such as water, ethanol, and the like. Unless otherwise indicated or inferred, it is intended that disclosed crystalline compounds include both solvated and unsolvated forms.

“Treating” includes any effect, e.g., lessening, reducing, modulating, or eliminating, that results in the improvement of the condition, disease, disorder and the like.

The term “disorder” refers to and is used interchangeably with, the terms “disease,” “condition,” or “illness,” unless otherwise indicated.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable excipient” or “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the present disclosure is desirably a mammal in which treatment, for example, of a cancer or a blood disorder is desired. “Modulation” includes antagonism (e.g., inhibition), agonism, partial antagonism and/or partial agonism.

In the present specification, the terms “effective amount” or “therapeutically effective amount” means the amount of the subject compound that will elicit the biological or medical response of a tissue, system or animal, (e.g. mammal or human) that is being sought by the researcher, veterinarian, medical doctor or other clinician. The compounds of the present disclosure are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of basic groups that may be present in compounds used in the compositions. Compounds included in the present compositions that are basic in nature are capable of forming a wide variety of salts with various inorganic and organic acids.

The term “and/or” is used in this disclosure to mean either “and” or “or” unless indicated otherwise.

As used herein, the words “a” and “an” are meant to include one or more unless otherwise specified. For example, the term “an agent” encompasses both a single agent and a combination of two or more agents.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. The term “about” in the context of peaks at degrees 2θ means that there is an uncertainty in the measurements of the 2θ of ±0.2 (expressed in 2θ). Generally, a DSC thermogram may have a variation in the range of ±2° C. Therefore, the temperature values should be understood as including values in the range of about: 2° C.

In general, provided herein are crystalline salt forms of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide that are substantially free of any other crystalline forms, unless indicated otherwise. As used herein, “substantially free” or substantially free of any other crystalline forms” means that the disclosed crystalline form contains about 20% or less, about 10% or less, about 5% or less, about 2% or less, or about 1% or less, of any other crystalline forms of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide as measured, for example, by XRPD, or less than about 20%, less than about 10%, less than about 5%, less than about 4%, less than about 3%, less than about 2% or less than about 1%, of any other crystalline forms of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide as measured, for example, by XRPD. Thus, a disclosed crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide described herein as substantially free of any other crystalline forms would be understood to contain greater than 80% (w/w), greater than 90% (w/w), greater than 95% (w/w), greater than 98% (w/w), or greater than 99% (w/w) of the said crystalline forms of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide. Accordingly, in some embodiments, a disclosed crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide may contain from 1% to 20% (w/w), from 5% to 20% (w/w), or from 5% to 10% (w/w) of one or more other crystalline forms of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide.

### Crystalline Forms

The present disclosure is directed, at least in part, to crystalline salt forms of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide. Also disclosed are crystalline hydrates, anhydrates, hemihydrates, solvates, tautomers and cocrystals of any of the crystalline forms described herein.

For example, disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, characterized by a powder X-ray diffraction pattern having a characteristic peak in degrees 2θ at about 24.0 (referred to herein as “Form 1-1”).

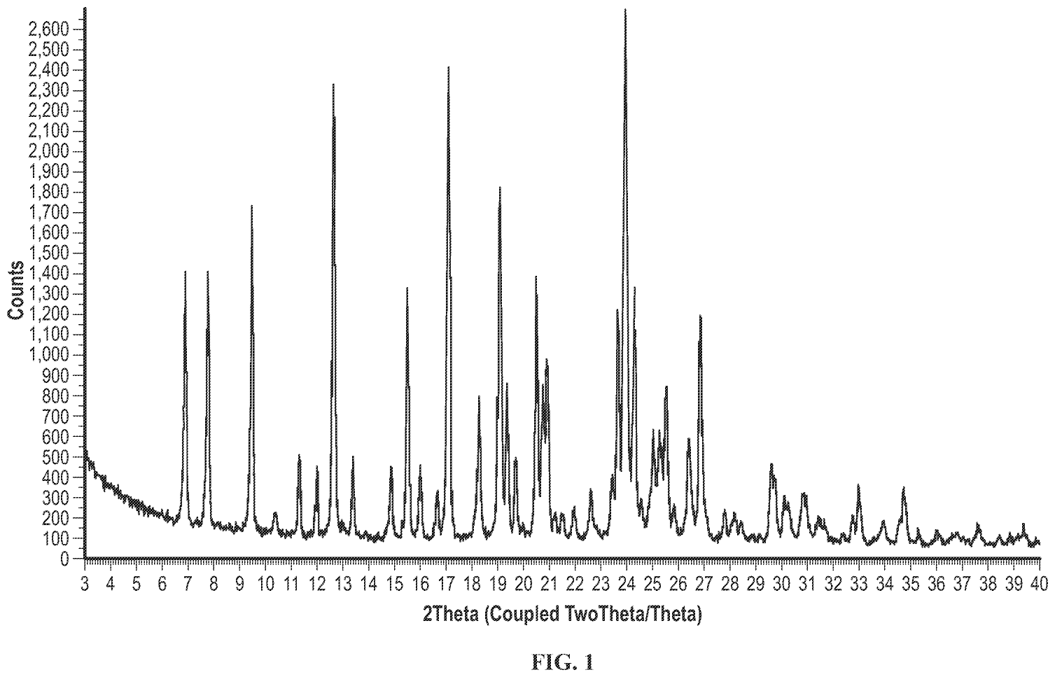

In one embodiment, the crystalline Form 1-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropane carboxamide, methanesulfonic acid salt, is characterized by a powder X-ray diffraction pattern that has a characteristic peak in degrees 2θ at about 6.9, is characterized by a powder X-ray diffraction pattern that has a characteristic peak in degrees 2θ at about 7.8, is characterized by a powder X-ray diffraction pattern that has a characteristic peak in degrees 2θ at about 9.5, is characterized by a powder X-ray diffraction pattern that has a characteristic peak in degrees 2θ at about 12.7, is characterized by a powder X-ray diffraction pattern that has a characteristic peak in degrees 2θ at about 15.5, is characterized by a powder X-ray diffraction pattern that has a characteristic peak in degrees 2θ at about 17.1, is characterized by a powder X-ray diffraction pattern that has a characteristic peak in degrees 2θ at about 19.1, is characterized by a powder X-ray diffraction pattern that has a characteristic peak in degrees 2θ at about 19.4, is characterized by a powder X-ray diffraction pattern that has a characteristic peak in degrees 2θ at about 20.5, is characterized by a powder X-ray diffraction pattern that has a characteristic peak in degrees 2θ at about 20.8, is characterized by a powder X-ray diffraction pattern that has a characteristic peak in degrees 2θ at about 21.0, is characterized by a powder X-ray diffraction pattern that has a characteristic peak in degrees 2θ at about 23.7, is characterized by a powder X-ray diffraction pattern that has a characteristic peak in degrees 2θ at about 24.0, is characterized by a powder X-ray diffraction pattern that has a characteristic peak in degrees 2θ at about 24.3, is characterized by a powder X-ray diffraction pattern that has a characteristic peak in degrees 2θ at about 25.5, and/or is characterized by a powder X-ray diffraction pattern that has a characteristic peak in degrees 2θ at about 26.9. In another embodiment, crystalline Form 1-1 is characterized by a powder X-ray diffraction pattern having at least one or more characteristic peaks in degrees 2θ at about 12.7, 17.1, and 24.0. In a further embodiment, crystalline Form 1-1 is characterized by a powder X-ray diffraction pattern having at least one or more characteristic peaks in degrees 2θ at about 9.5, 12.7, 17.1, 19.1, 20.5, and 24.0. In yet another embodiment, crystalline Form 1-1 is characterized by a powder X-ray diffraction pattern having at least one or more characteristic peaks in degrees 2θ at about 9.5, 12.7, 15.5, 17.1, 19.1, 20.5, 24.0, 24.3, and 26.9. In another embodiment, crystalline Form 1-1 is characterized by a powder X-ray diffraction pattern having at least one or more characteristic peaks in degrees 2θ at about 6.9, 7.8, 9.5, 12.7, 15.5, 17.1, 19.1, 20.5, 21.0, 23.7, 24.0, 24.3, and 26.9. In another embodiment, crystalline Form 1-1 is characterized by a powder X-ray diffraction pattern having at least one or more characteristic peaks in degrees 2θ at about 6.9, 7.8, 9.5, 12.7, 15.5, 17.1, 19.1, 19.4, 20.5, 20.8, 21.0, 23.7, 24.0, 24.3, 25.5, and 26.9. For example, a contemplated crystalline form has a powder X-ray diffraction pattern shown in FIG. 1. In one embodiment, the powder X-ray diffraction pattern of the crystalline form was obtained using Cu Kat radiation.

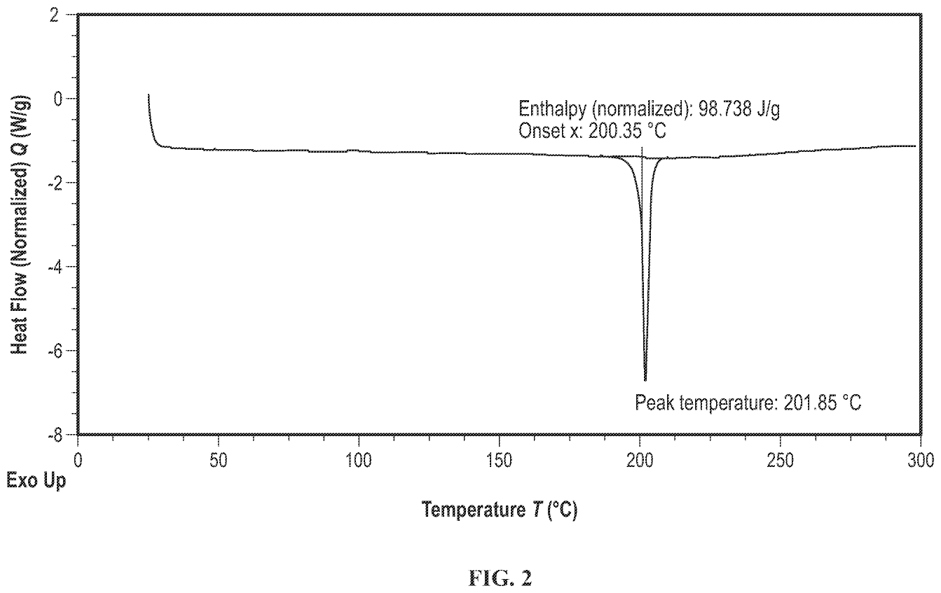

The contemplated crystalline Form 1-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, may be characterized by a differential scanning calorimetry (DSC) profile showing a characteristic endotherm with an onset of about 200° C. and a peak of about 202° C. (enthalpy 98.7 J/g). Form 1-1, for example, may be characterized by the differential scanning calorimetry profile shown in FIG. 2.

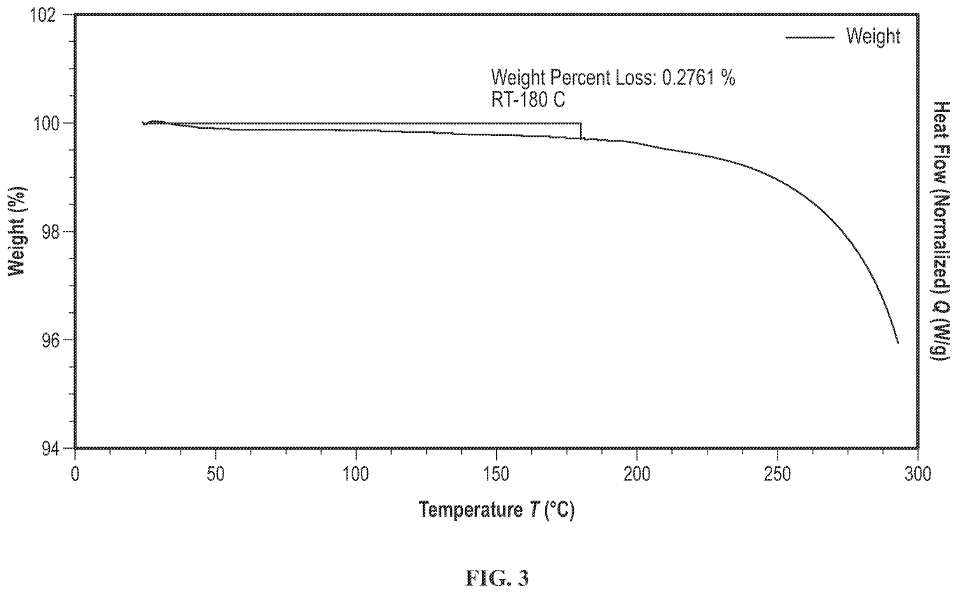

The contemplated crystalline Form 1-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, may be characterized by a thermogravimetric analysis (TGA) profile showing a mass loss of about 0.27 wt. % between about 20° C. to about 180° C. Form 1-1, for example, may be characterized by the thermogravimetric analysis profile shown in FIG. 3.

In some embodiments, crystalline Form 1-1 may be characterized by a dynamic vapor sorption (DVS) profile showing a reversable total mass change of about 0.6 wt. % between about 0% to about 80% relative humidity (RH) at 25° C., and a reversable total mass change of about 1.3 wt. % between about 0% to about 90% relative humidity (RH) at 25° C. In certain embodiments, the contemplated crystalline Form 1-1 is an anhydrous crystalline form.

In another embodiment, disclosed herein is a different crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, characterized by a powder X-ray diffraction pattern having a characteristic peak in degrees 2θ at about 9.9 (referred to herein as “Form 1-2”).

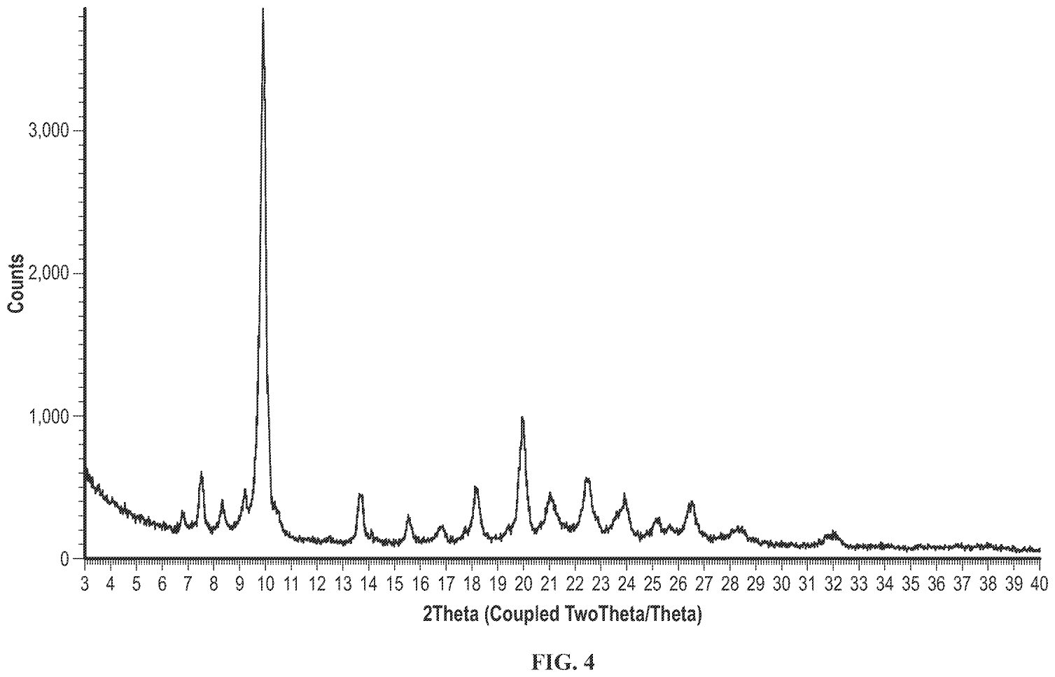

In one embodiment, the crystalline Form 1-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, is characterized by a powder X-ray diffraction pattern that has a characteristic peak in degrees 2θ at about 7.5, is characterized by a powder X-ray diffraction pattern that has a characteristic peak in degrees 2θ at about 8.3, is characterized by a powder X-ray diffraction pattern that has a characteristic peak in degrees 2θ at about 9.2, is characterized by a powder X-ray diffraction pattern that has a characteristic peak in degrees 2θ at about 9.9, is characterized by a powder X-ray diffraction pattern that has a characteristic peak in degrees 2θ at about 10.4, is characterized by a powder X-ray diffraction pattern that has a characteristic peak in degrees 2θ at about 13.7, is characterized by a powder X-ray diffraction pattern that has a characteristic peak in degrees 2θ at about 18.2, is characterized by a powder X-ray diffraction pattern that has a characteristic peak in degrees 2θ at about 20.0, is characterized by a powder X-ray diffraction pattern that has a characteristic peak in degrees 2θ at about 21.1, is characterized by a powder X-ray diffraction pattern that has a characteristic peak in degrees 2θ at about 23.9, is characterized by a powder X-ray diffraction pattern that has a characteristic peak in degrees 2θ at about 22.2, and/or is characterized by a powder X-ray diffraction pattern that has a characteristic peak in degrees 2θ at about 26.5. In one embodiment, crystalline Form 1-2 is characterized by a powder X-ray diffraction pattern having at least one or more characteristic peaks in degrees 2θ at about 7.5, 9.9, and 20.0. In another embodiment, crystalline Form 1-2 is characterized by a powder X-ray diffraction pattern having at least one or more characteristic peaks in degrees 2θ at about 7.5, 9.9, 13.7, 18.2, 20.0, and 22.5. In yet another embodiment, crystalline Form 1-2 is characterized by a powder X-ray diffraction pattern having at least one or more characteristic peaks in degrees 2θ at about 7.5, 9.2, 9.9, 13.7, 18.2, 20.0, 23.9, 22.5, and 26.5. In a further embodiment, crystalline Form 1-2 is characterized by a powder X-ray diffraction pattern having at least one or more characteristic peaks in degrees 2θ at about 7.5, 8.3, 9.2, 9.9, 10.4, 13.7, 18.2, 20.0, 21.1, 23.9, 22.5, and 26.5. For example, a contemplated crystalline form has a powder X-ray diffraction pattern shown in FIG. 4. In one embodiment, the powder X-ray diffraction pattern of the crystalline form was obtained using Cu Kα radiation.

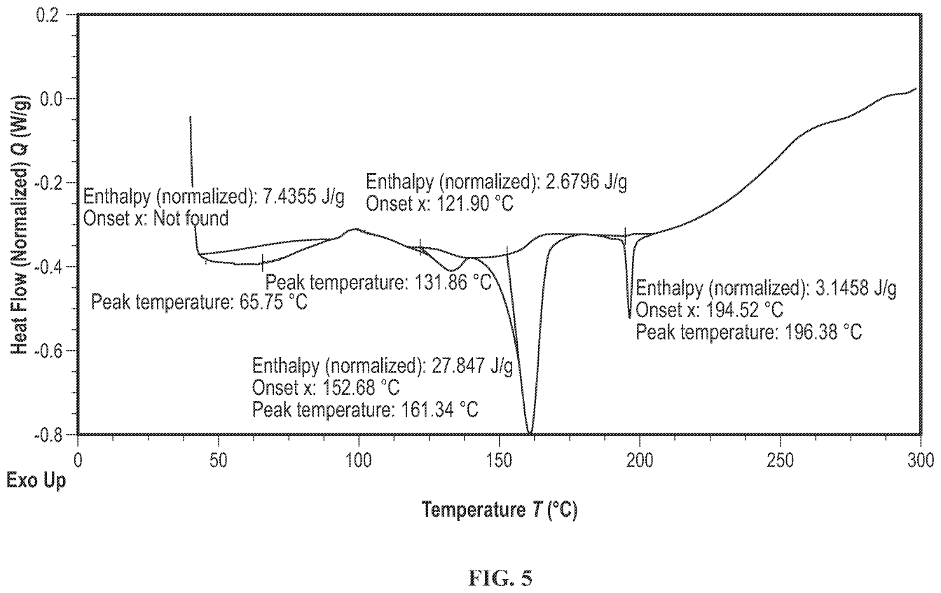

The contemplated crystalline Form 1-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, may be characterized by a differential scanning calorimetry (DSC) profile showing a characteristic endotherm with a peak of about 66° C. (enthalpy 7.45 J/g), a characteristic endotherm with an onset of about 122° C. and a peak of about 132° C. (enthalpy 2.68 J/g), a characteristic endotherm with an onset of about 153° C. and a peak of about 161° C. (enthalpy 27.8 J/g), and a characteristic endotherm with an onset of about 195° C. and a peak of about 195° C. (enthalpy 3.15 J/g). Form 1-2, for example, may be characterized by the differential scanning calorimetry profile shown in FIG. 5.

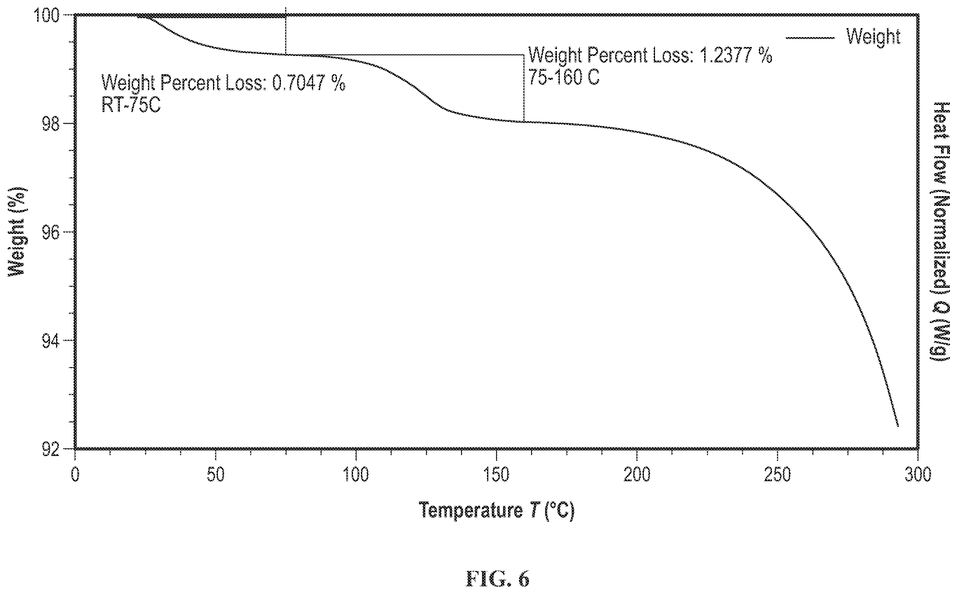

The contemplated crystalline Form 1-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, may be characterized by a thermogravimetric analysis (TGA) profile showing a mass loss of about 0.70 wt. % between about 20° C. to about 75° C., and a mass loss of about 1.24 wt. % between about 75° C. to about 160° C. Form 1-2, for example, may be characterized by the thermogravimetric analysis profile shown in FIG. 6. In certain embodiments, the contemplated crystalline Form 1-2 is a tetrahydrofuran solvate (0.1 mol).

In another embodiment, disclosed herein is a different crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, characterized by a powder X-ray diffraction pattern having a characteristic peak in degrees 2θ at about 21.0 (referred to herein as “Form 1-3”).

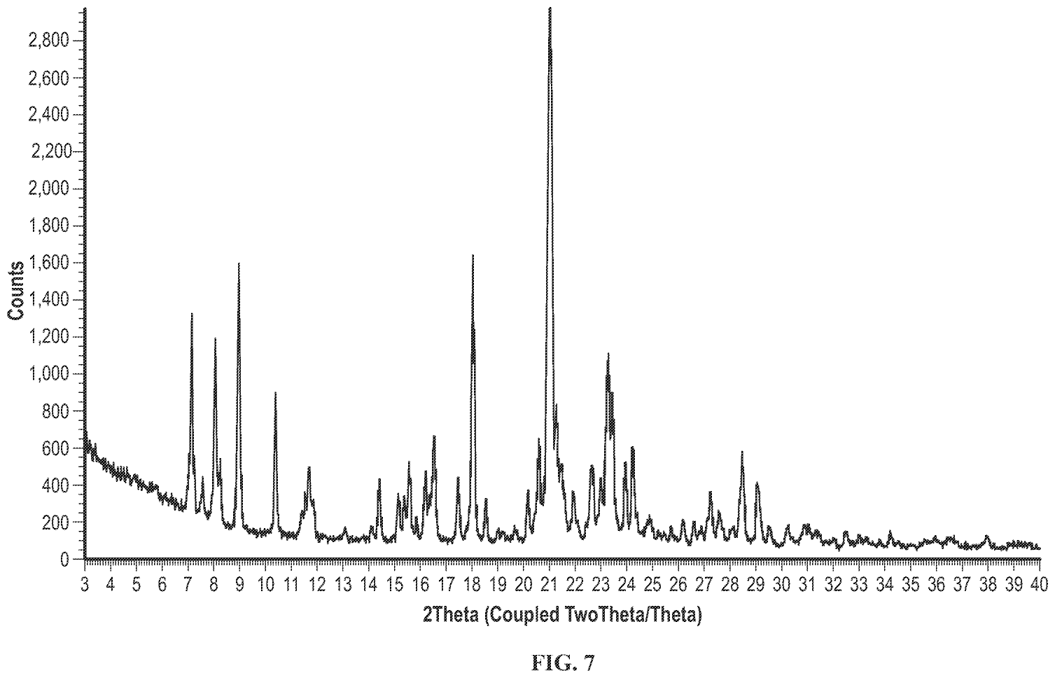

In one embodiment, the crystalline Form 1-3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropane carboxamide, methanesulfonic acid salt, is characterized by a powder X-ray diffraction pattern that has a characteristic peak in degrees 2θ at about 7.1, is characterized by a powder X-ray diffraction pattern that has a characteristic peak in degrees 2θ at about 8.1, is characterized by a powder X-ray diffraction pattern that has a characteristic peak in degrees 2θ at about 9.0, is characterized by a powder X-ray diffraction pattern that has a characteristic peak in degrees 2θ at about 10.4, is characterized by a powder X-ray diffraction pattern that has a characteristic peak in degrees 2θ at about 11.1, is characterized by a powder X-ray diffraction pattern that has a characteristic peak in degrees 2θ at about 15.6, is characterized by a powder X-ray diffraction pattern that has a characteristic peak in degrees 2θ at about 16.5, is characterized by a powder X-ray diffraction pattern that has a characteristic peak in degrees 2θ at about 17.5, is characterized by a powder X-ray diffraction pattern that has a characteristic peak in degrees 2θ at about 18.0, is characterized by a powder X-ray diffraction pattern that has a characteristic peak in degrees 2θ at about 20.6, is characterized by a powder X-ray diffraction pattern that has a characteristic peak in degrees 2θ at about 21.0, is characterized by a powder X-ray diffraction pattern that has a characteristic peak in degrees 2θ at about 23.3, is characterized by a powder X-ray diffraction pattern that has a characteristic peak in degrees 2θ at about 24.0, is characterized by a powder X-ray diffraction pattern that has a characteristic peak in degrees 2θ at about 24.2, and/or is characterized by a powder X-ray diffraction pattern that has a characteristic peak in degrees 2θ at about 28.5. In another embodiment, crystalline Form 1-3 is characterized by a powder X-ray diffraction pattern having at least one or more characteristic peaks in degrees 2θ at about 9.0, 18.0, and 21.0. In a further embodiment, crystalline Form 1-3 is characterized by a powder X-ray diffraction pattern having at least one or more characteristic peaks in degrees 2θ at about 7.1, 8.1, 9.0, 18.0, 21.0, and 23.3. In yet another embodiment, crystalline Form 1-3 is characterized by a powder X-ray diffraction pattern having at least one or more characteristic peaks in degrees 2θ at about 7.1, 8.1, 9.0, 10.4, 18.0, 20.6, 21.0, 23.3, and 28.5. In another embodiment, crystalline Form 1-3 is characterized by a powder X-ray diffraction pattern having at least one or more characteristic peaks in degrees 2θ at about 7.1, 8.1, 9.0, 10.4, 16.5, 18.0, 20.6, 21.0, 23.3, 24.0, 24.2, and 28.5. In another embodiment, crystalline Form 1-3 is characterized by a powder X-ray diffraction pattern having at least one or more characteristic peaks in degrees 2θ at about 7.1, 8.1, 9.0, 10.4, 11.1, 15.6, 16.5, 17.5, 18.0, 20.6, 21.0, 23.3, 24.0, 24.2, and 28.5. For example, a contemplated crystalline form has a powder X-ray diffraction pattern shown in FIG. 7. In one embodiment, the powder X-ray diffraction pattern of the crystalline form was obtained using Cu Kat radiation.

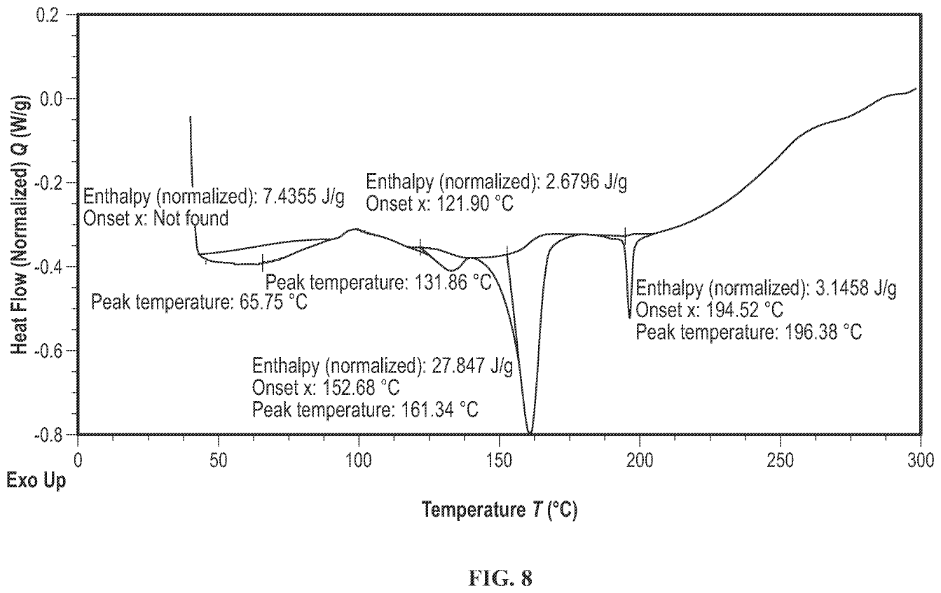

The contemplated crystalline Form 1-3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, may be characterized by a differential scanning calorimetry (DSC) profile showing a characteristic endotherm with an onset of about 208° C. and a peak of about 209° C. (enthalpy 92.0 J/g). Form 1-3, for example, may be characterized by the differential scanning calorimetry profile shown in FIG. 8.

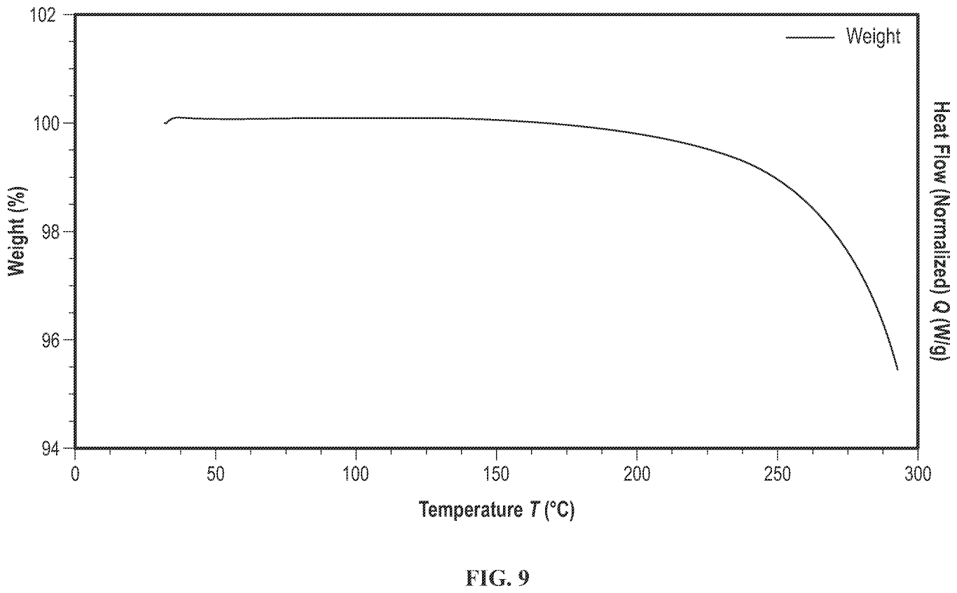

The contemplated crystalline Form 1-3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, may be characterized by a thermogravimetric analysis (TGA) profile showing no mass loss (0% wt.) between about 20° C. to about 150° C. Form 1-3, for example, may be characterized by the thermogravimetric analysis profile shown in FIG. 9. In some embodiments, the contemplated crystalline Form 1-3 is an anhydrous crystalline form.

In another embodiment, disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, fumaric acid salt, characterized by a powder X-ray diffraction pattern having a characteristic peak in degrees 2θ at about 26.1 (referred to herein as “Form 2”).

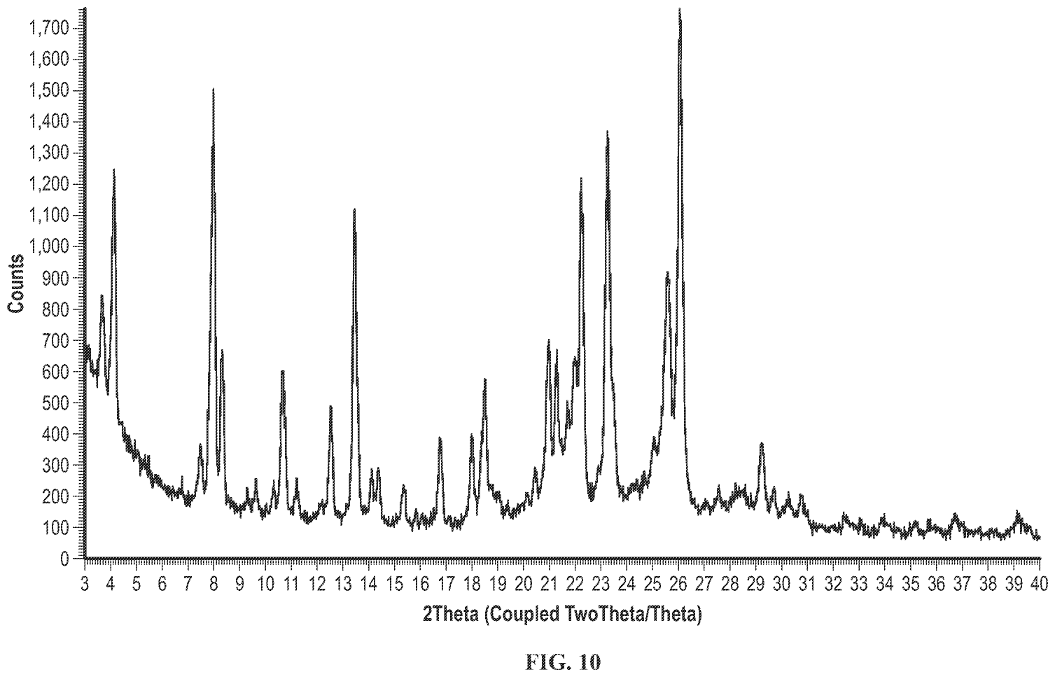

In one embodiment, the crystalline Form 2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropane carboxamide, fumaric acid salt, is characterized by a powder X-ray diffraction pattern that has a characteristic peak in degrees 2θ at about 4.2, is characterized by a powder X-ray diffraction pattern that has a characteristic peak in degrees 2θ at about 8.0, is characterized by a powder X-ray diffraction pattern that has a characteristic peak in degrees 2θ at about 8.3, is characterized by a powder X-ray diffraction pattern that has a characteristic peak in degrees 2θ at about 10.7, is characterized by a powder X-ray diffraction pattern that has a characteristic peak in degrees 2θ at about 12.5, is characterized by a powder X-ray diffraction pattern that has a characteristic peak in degrees 2θ at about 13.5, is characterized by a powder X-ray diffraction pattern that has a characteristic peak in degrees 2θ at about 18.5, is characterized by a powder X-ray diffraction pattern that has a characteristic peak in degrees 2θ at about 21.0, is characterized by a powder X-ray diffraction pattern that has a characteristic peak in degrees 2θ at about 22.0, is characterized by a powder X-ray diffraction pattern that has a characteristic peak in degrees 2θ at about 21.3, is characterized by a powder X-ray diffraction pattern that has a characteristic peak in degrees 2θ at about 21.7, is characterized by a powder X-ray diffraction pattern that has a characteristic peak in degrees 2θ at about 22.3, is characterized by a powder X-ray diffraction pattern that has a characteristic peak in degrees 2θ at about 23.3, is characterized by a powder X-ray diffraction pattern that has a characteristic peak in degrees 2θ at about 25.6, and/or is characterized by a powder X-ray diffraction pattern that has a characteristic peak in degrees 2θ at about 26.1. In another embodiment, crystalline Form 2 is characterized by a powder X-ray diffraction pattern having at least one or more characteristic peaks in degrees 2θ at about 8.0, 23.3, and 26.1. In a further embodiment, crystalline Form 2 is characterized by a powder X-ray diffraction pattern having at least one or more characteristic peaks in degrees 2θ at about 8.0, 13.5, 22.3, 23.3, 25.6, and 26.1. In yet another embodiment, crystalline Form 2 is characterized by a powder X-ray diffraction pattern having at least one or more characteristic peaks in degrees 2θ at about 4.2, 8.0, 8.3, 13.5, 21.0, 22.3, 23.3, 25.6, and 26.1. In another embodiment, crystalline Form 2 is characterized by a powder X-ray diffraction pattern having at least one or more characteristic peaks in degrees 2θ at about 4.2, 8.0, 8.3, 10.7, 13.5, 18.5, 21.0, 21.3, 22.3, 23.3, 25.6, and 26.1. In another embodiment, crystalline Form 2 is characterized by a powder X-ray diffraction pattern having at least one or more characteristic peaks in degrees 2θ at about 4.2, 8.0, 8.3, 10.7, 12.5, 13.5, 18.5, 21.0, 22.0, 21.3, 21.7, 22.3, 23.3, 25.6, and 26.1. For example, a contemplated crystalline form has a powder X-ray diffraction pattern shown in FIG. 10. In one embodiment, the powder X-ray diffraction pattern of the crystalline form was obtained using Cu Kα radiation.

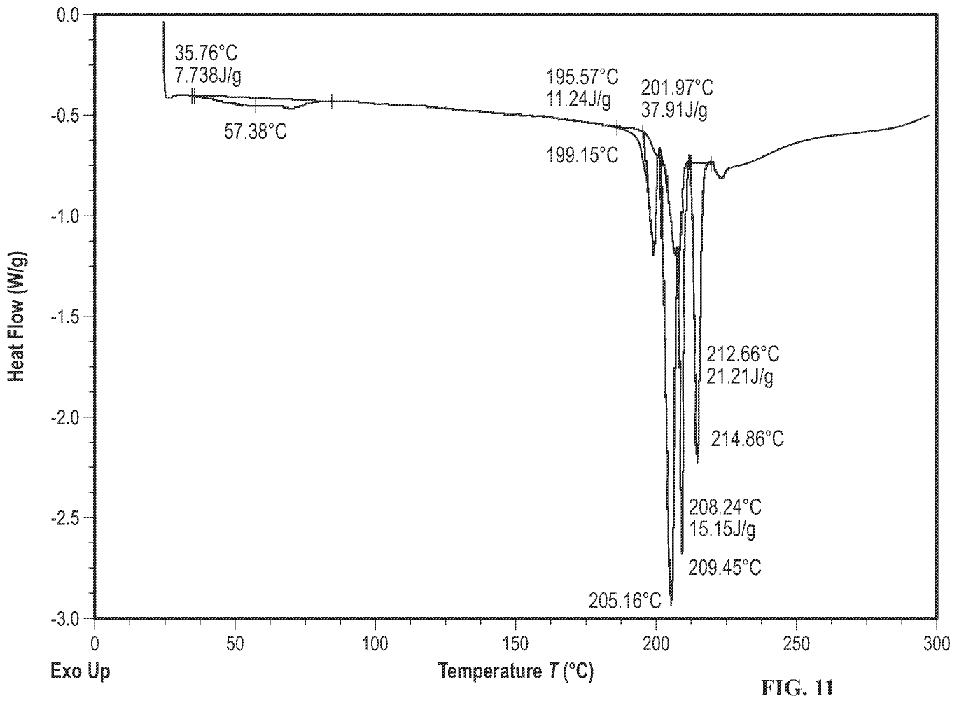

The contemplated crystalline Form 2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, fumaric acid salt, may be characterized by a differential scanning calorimetry (DSC) profile showing a characteristic endotherm with an onset of about 36° C. and a peak of about 57° C. (enthalpy 7.74 J/g), a characteristic endotherm with an onset of about 196° C. and a peak of about 199° C. (enthalpy 11.2 J/g), a characteristic endotherm with an onset of about 202° C. and a peak of about 205° C. (enthalpy 37.9 J/g), a characteristic endotherm with an onset of about 208° C. and a peak of about 209° C. (enthalpy 15.1 J/g), and a characteristic endotherm with an onset of about 213° C. and a peak of about 215° C. (enthalpy 21.2 J/g). Form 2, for example, may be characterized by the differential scanning calorimetry profile shown in FIG. 11.

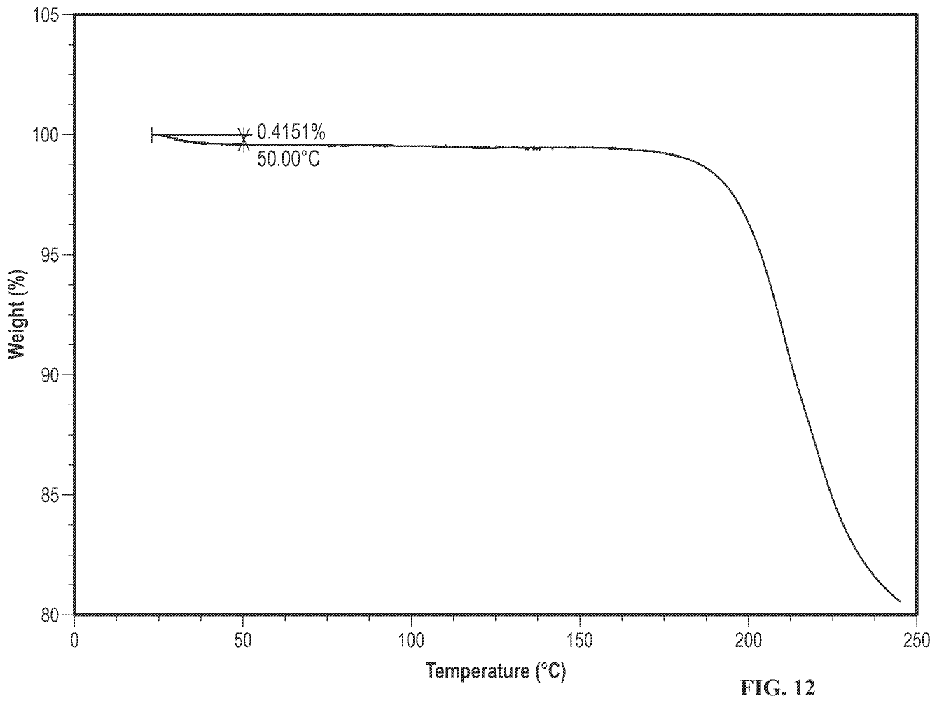

The contemplated crystalline Form 2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, fumaric acid salt, may be characterized by a thermogravimetric analysis (TGA) profile showing a mass loss of about 0.42 wt. % between about 20° C. to about 50 CC. Form 2, for example, may be characterized by the thermogravimetric analysis profile shown in FIG. 12. In certain embodiments, the contemplated crystalline Form 2 is an anhydrous crystalline form.

In another embodiment, disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, hydrogen bromide salt, characterized by a powder X-ray diffraction pattern having a characteristic peak in degrees 2θ at about 9.7 (referred to herein as “Form 3”).

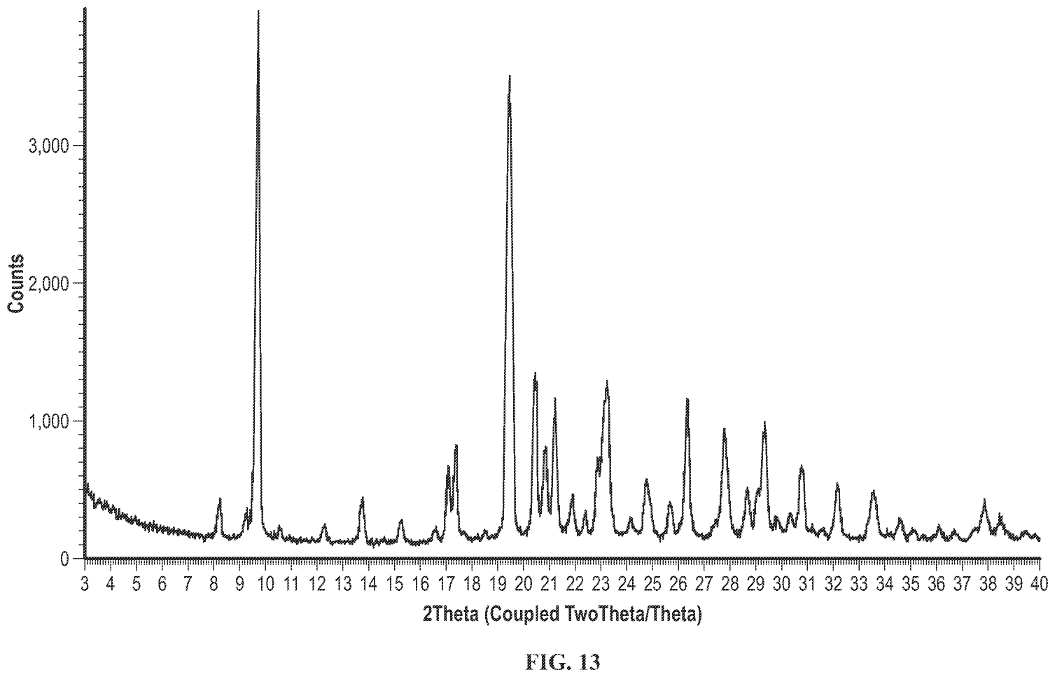

In one embodiment, the crystalline Form 3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropane carboxamide, hydrogen bromide salt, is characterized by a powder X-ray diffraction pattern that has a characteristic peak in degrees 2θ at about 9.7, is characterized by a powder X-ray diffraction pattern that has a characteristic peak in degrees 2θ at about 17.1, is characterized by a powder X-ray diffraction pattern that has a characteristic peak in degrees 2θ at about 17.4, is characterized by a powder X-ray diffraction pattern that has a characteristic peak in degrees 2θ at about 19.5, is characterized by a powder X-ray diffraction pattern that has a characteristic peak in degrees 2θ at about 20.5, is characterized by a powder X-ray diffraction pattern that has a characteristic peak in degrees 2θ at about 20.9, is characterized by a powder X-ray diffraction pattern that has a characteristic peak in degrees 2θ at about 21.2, is characterized by a powder X-ray diffraction pattern that has a characteristic peak in degrees 2θ at about 22.9, is characterized by a powder X-ray diffraction pattern that has a characteristic peak in degrees 2θ at about 23.3, is characterized by a powder X-ray diffraction pattern that has a characteristic peak in degrees 2θ at about 24.8, is characterized by a powder X-ray diffraction pattern that has a characteristic peak in degrees 2θ at about 26.4, is characterized by a powder X-ray diffraction pattern that has a characteristic peak in degrees 2θ at about 27.8, is characterized by a powder X-ray diffraction pattern that has a characteristic peak in degrees 2θ at about 29.4, is characterized by a powder X-ray diffraction pattern that has a characteristic peak in degrees 2θ at about 30.8, and/or is characterized by a powder X-ray diffraction pattern that has a characteristic peak in degrees 2θ at about 32.2. In another embodiment, crystalline Form 3 is characterized by a powder X-ray diffraction pattern having at least one or more characteristic peaks in degrees 2θ at about 9.7, 19.5, and 20.5. In a further embodiment, crystalline Form 3 is characterized by a powder X-ray diffraction pattern having at least one or more characteristic peaks in degrees 2θ at about 9.7, 19.5, 20.5, 21.2, 23.3, and 26.4. In yet another embodiment, crystalline Form 3 is characterized by a powder X-ray diffraction pattern having at least one or more characteristic peaks in degrees 2θ at about 9.7, 17.4, 19.5, 20.5, 21.2, 23.3, 26.4, 27.8, and 29.4. In another embodiment, crystalline Form 3 is characterized by a powder X-ray diffraction pattern having at least one or more characteristic peaks in degrees 2θ at about 9.7, 17.1, 17.4, 19.5, 20.5, 20.9, 21.2, 22.9, 23.3, 26.4, 27.8, and 29.4. In another embodiment, crystalline Form 3 is characterized by a powder X-ray diffraction pattern having at least one or more characteristic peaks in degrees 2θ at about 9.7, 17.1, 17.4, 19.5, 20.5, 20.9, 21.2, 22.9, 23.3, 24.8, 26.4, 27.8, 29.4, 30.8, and 32.2. For example, a contemplated crystalline form has a powder X-ray diffraction pattern shown in FIG. 13. In one embodiment, the powder X-ray diffraction pattern of the crystalline form was obtained using Cu Kat radiation.

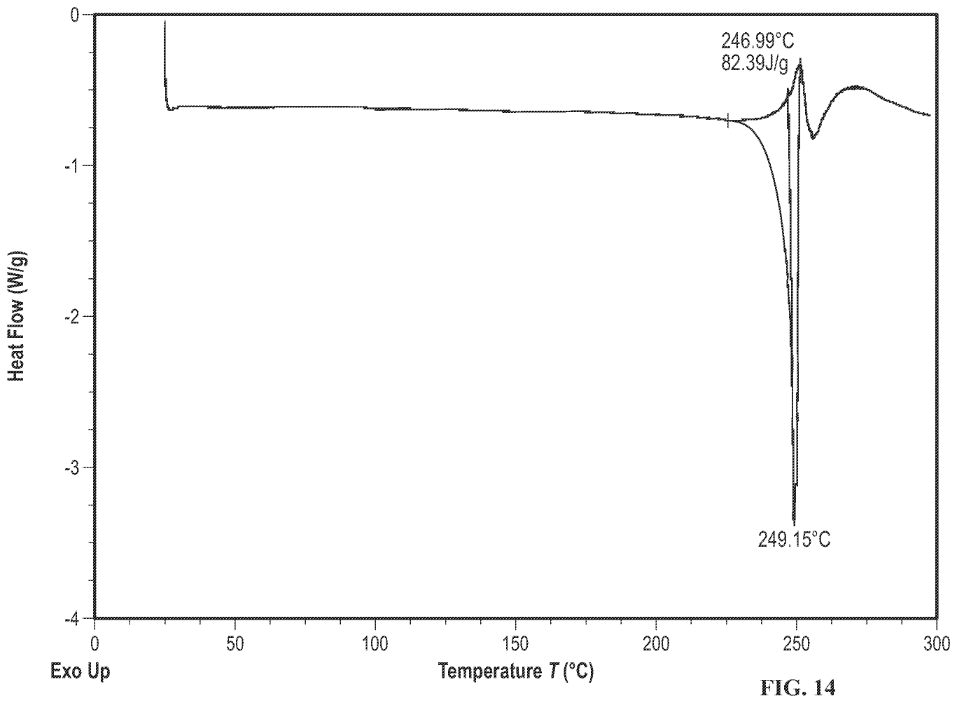

The contemplated crystalline Form 3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, hydrogen bromide salt, may be characterized by a differential scanning calorimetry (DSC) profile showing a characteristic endotherm with an onset of about 247° C. and a peak of about 249° C. (enthalpy 82.4 J/g). Form 3, for example, may be characterized by the differential scanning calorimetry profile shown in FIG. 14.

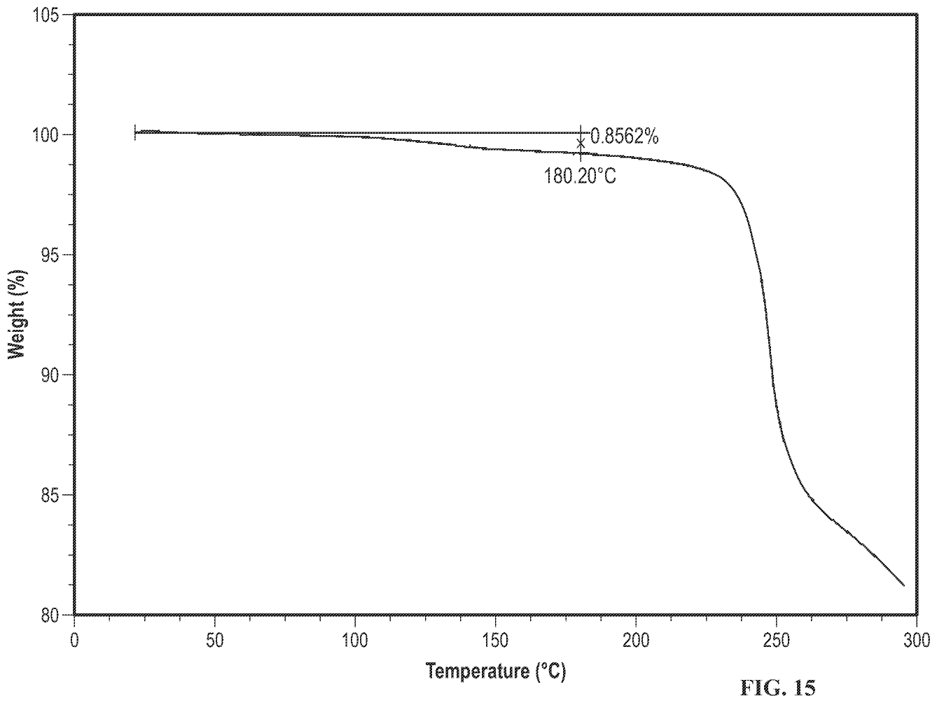

The contemplated crystalline Form 3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, hydrogen bromide salt, may be characterized by a thermogravimetric analysis (TGA) profile showing a mass loss of about 0.86 wt. % between about 20° C. to about 180° C. Form 3, for example, may be characterized by the thermogravimetric analysis profile shown in FIG. 15. In certain embodiments, the contemplated crystalline Form 3 is an anhydrous crystalline form.

In another embodiment, disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, phosphoric acid salt, characterized by a powder X-ray diffraction pattern having a characteristic peak in degrees 2θ at about 4.8 (referred to herein as “Form 4”).

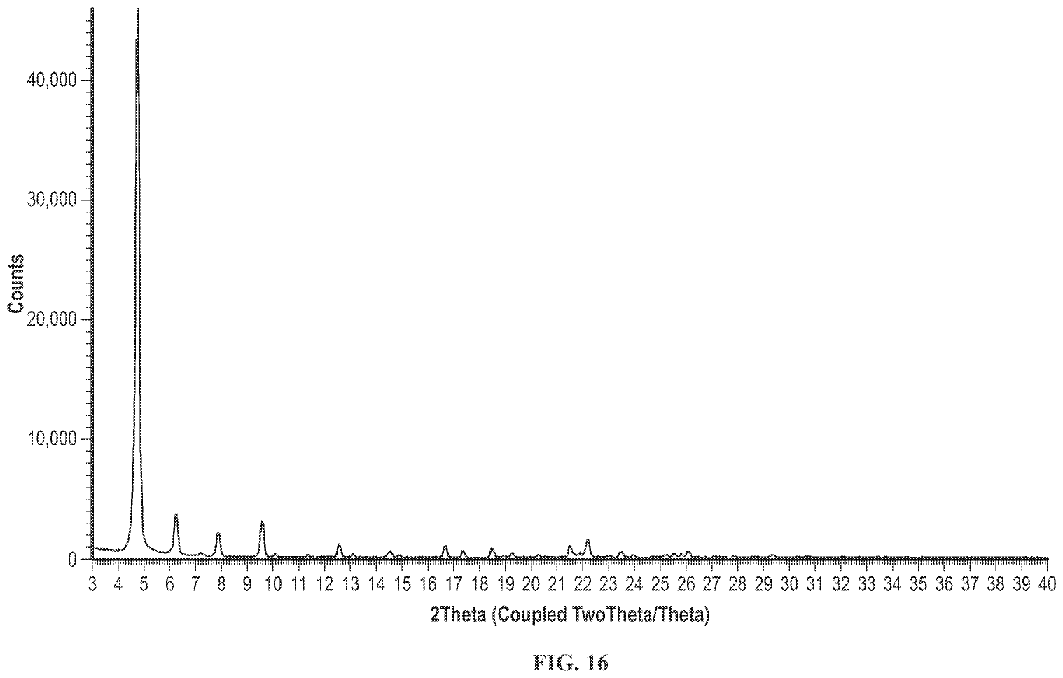

In one embodiment, the crystalline Form 4 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropane carboxamide, phosphoric acid salt, is characterized by a powder X-ray diffraction pattern that has a characteristic peak in degrees 2θ at about 4.8, is characterized by a powder X-ray diffraction pattern that has a characteristic peak in degrees 2θ at about 6.2, is characterized by a powder X-ray diffraction pattern that has a characteristic peak in degrees 2θ at about 7.9, is characterized by a powder X-ray diffraction pattern that has a characteristic peak in degrees 2θ at about 9.6, is characterized by a powder X-ray diffraction pattern that has a characteristic peak in degrees 2θ at about 12.6, is characterized by a powder X-ray diffraction pattern that has a characteristic peak in degrees 2θ at about 16.7, is characterized by a powder X-ray diffraction pattern that has a characteristic peak in degrees 2θ at about 18.5, is characterized by a powder X-ray diffraction pattern that has a characteristic peak in degrees 2θ at about 21.5, and/or is characterized by a powder X-ray diffraction pattern that has a characteristic peak in degrees 2θ at about 22.2. In another embodiment, crystalline Form 4 is characterized by a powder X-ray diffraction pattern having at least one or more characteristic peaks in degrees 2θ at about 4.8, 6.2, and 9.6. In a further embodiment, crystalline Form 4 is characterized by a powder X-ray diffraction pattern having at least one or more characteristic peaks in degrees 2θ at about 4.8, 6.2, 7.9, 9.6, 12.6, and 22.2. In yet another embodiment, crystalline Form 4 is characterized by a powder X-ray diffraction pattern having at least one or more characteristic peaks in degrees 2θ at about 4.8, 6.2, 7.9, 9.6, 12.6, 16.7, 18.5, 21.5, and 22.2. For example, a contemplated crystalline form has a powder X-ray diffraction pattern shown in FIG. 16. In one embodiment, the powder X-ray diffraction pattern of the crystalline form was obtained using Cu Kat radiation.

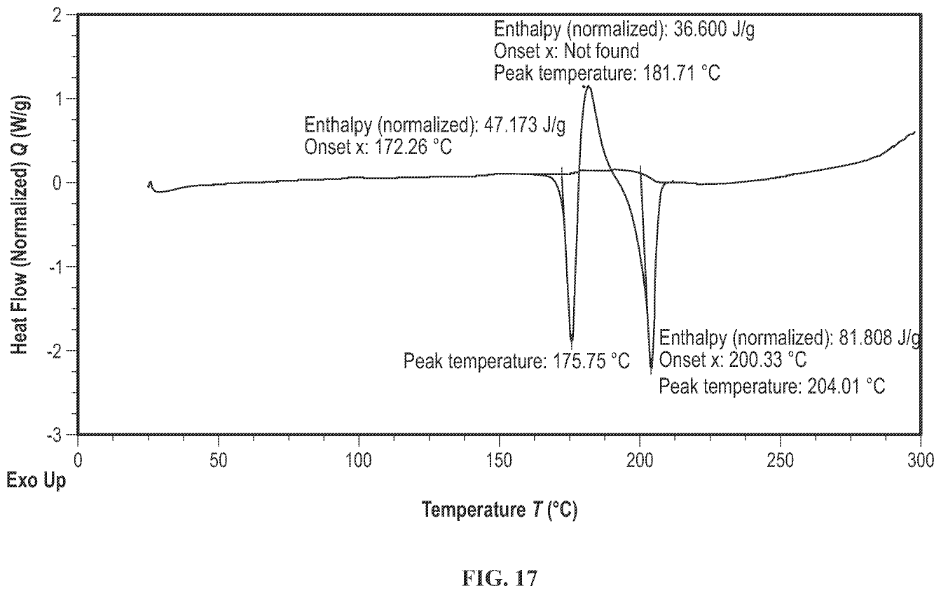

The contemplated crystalline Form 4 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, phosphoric salt, may be characterized by a differential scanning calorimetry (DSC) profile showing a characteristic endotherm with an onset of about 172° C. and a peak of about 275° C. (enthalpy 47.2 J/g), a characteristic exotherm with a peak of about 182° C. (enthalpy 36.6 J/g), and a characteristic endotherm with an onset of about 200° C. and a peak of about 204° C. (enthalpy 81.8 J/g). Form 4, for example, may be characterized by the differential scanning calorimetry profile shown in FIG. 17.

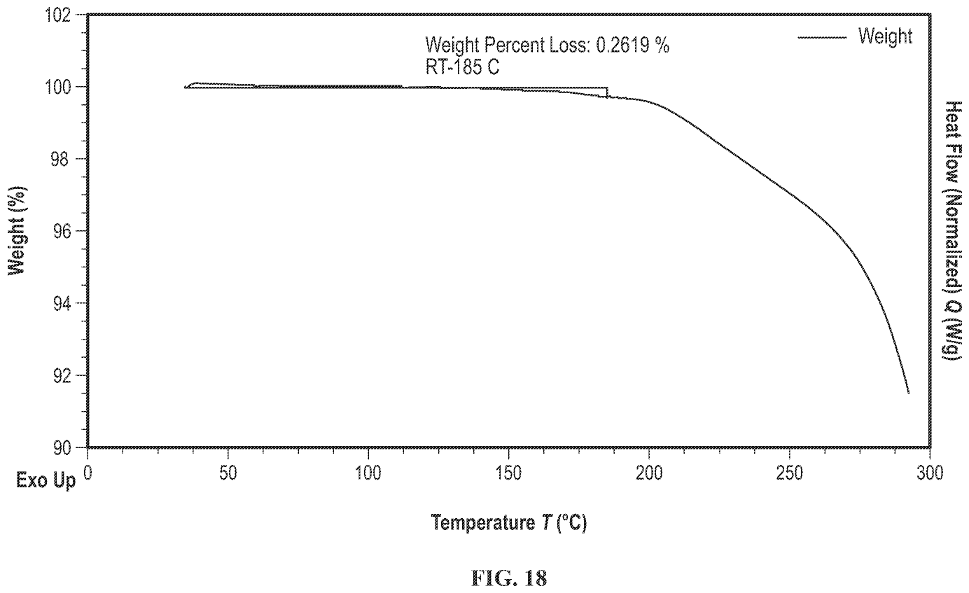

The contemplated crystalline Form 4 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, phosphoric acid salt, may be characterized by a thermogravimetric analysis (TGA) profile showing a mass loss of about 0.26 wt. % between about 20° C. to about 185° C. Form 4, for example, may be characterized by the thermogravimetric analysis profile shown in FIG. 18. In certain embodiments, the contemplated crystalline Form 4 is an anhydrous crystalline form.

In another embodiment, disclosed herein is a crystalline form of N-(4-((2-methoxy-3-(1-(methyl-d₃)-1H-1,2,4-triazol-3-yl)phenyl)amino)-5-(propanoyl-3,3, 3-d₃)pyridin-2-yl) cyclopropanecarboxamide, sulfuric acid salt, characterized by a powder X-ray diffraction pattern having a characteristic peak in degrees 2θ at about 21.2 (referred to herein as “Form 5-1”).

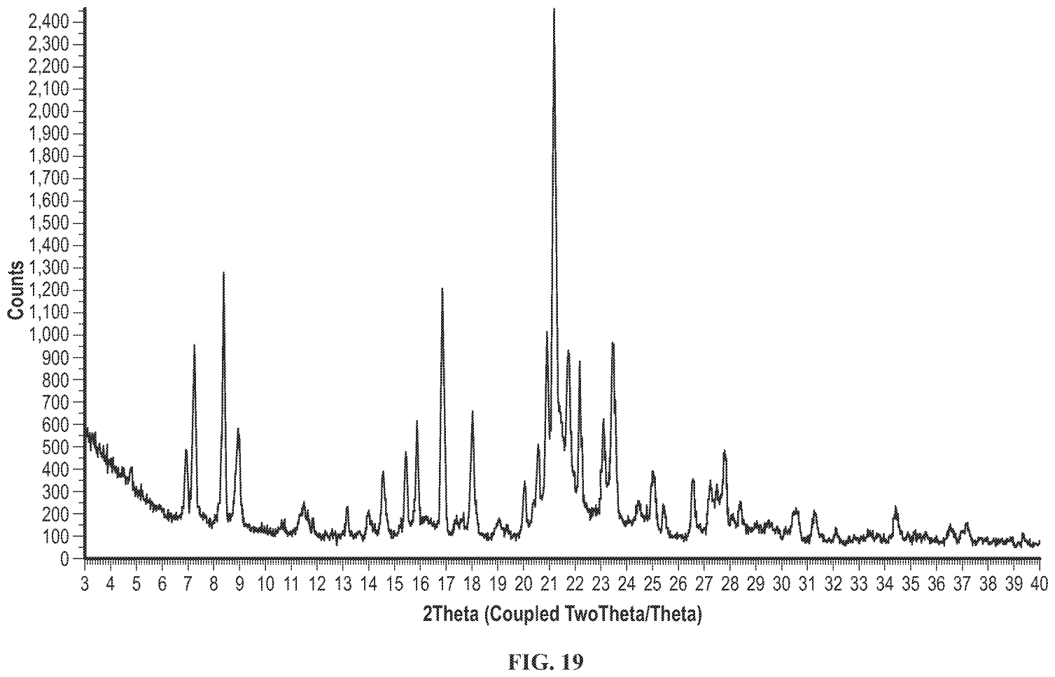

In one embodiment, the crystalline Form 5-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropane carboxamide, sulfuric acid salt, is characterized by a powder X-ray diffraction pattern that has a characteristic peak in degrees 2θ at about 7.3, is characterized by a powder X-ray diffraction pattern that has a characteristic peak in degrees 2θ at about 8.4, is characterized by a powder X-ray diffraction pattern that has a characteristic peak in degrees 2θ at about 9.0, is characterized by a powder X-ray diffraction pattern that has a characteristic peak in degrees 2θ at about 15.4, is characterized by a powder X-ray diffraction pattern that has a characteristic peak in degrees 2θ at about 15.9, is characterized by a powder X-ray diffraction pattern that has a characteristic peak in degrees 2θ at about 16.9, is characterized by a powder X-ray diffraction pattern that has a characteristic peak in degrees 2θ at about 18.0, is characterized by a powder X-ray diffraction pattern that has a characteristic peak in degrees 2θ at about 20.6, is characterized by a powder X-ray diffraction pattern that has a characteristic peak in degrees 2θ at about 20.9, is characterized by a powder X-ray diffraction pattern that has a characteristic peak in degrees 2θ at about 21.2, is characterized by a powder X-ray diffraction pattern that has a characteristic peak in degrees 2θ at about 21.8, is characterized by a powder X-ray diffraction pattern that has a characteristic peak in degrees 2θ at about 22.2, is characterized by a powder X-ray diffraction pattern that has a characteristic peak in degrees 2θ at about 23.1, is characterized by a powder X-ray diffraction pattern that has a characteristic peak in degrees 2θ at about 23.5, and/or is characterized by a powder X-ray diffraction pattern that has a characteristic peak in degrees 2θ at about 27.8. In another embodiment, crystalline Form 5-1 is characterized by a powder X-ray diffraction pattern having at least one or more characteristic peaks in degrees 2θ at about 8.4, 16.9, and 21.2. In a further embodiment, crystalline Form 5-1 is characterized by a powder X-ray diffraction pattern having at least one or more characteristic peaks in degrees 2θ at about 8.4, 16.9, 20.9, 21.2, 22.2, and 23.5. In yet another embodiment, crystalline Form 5-1 is characterized by a powder X-ray diffraction pattern having at least one or more characteristic peaks in degrees 2θ at about 7.3, 8.4, 16.9, 18.0, 20.9, 21.2, 21.8, 22.2, and 23.5. In another embodiment, crystalline Form 5-1 is characterized by a powder X-ray diffraction pattern having at least one or more characteristic peaks in degrees 2θ at about 7.3, 8.4, 9.0, 15.9, 16.9, 18.0, 20.9, 21.2, 21.8, 22.2, 23.1, and 23.5. In another embodiment, crystalline Form 5-1 is characterized by a powder X-ray diffraction pattern having at least one or more characteristic peaks in degrees 2θ at about 7.3, 8.4, 9.0, 15.4, 15.9, 16.9, 18.0, 20.6, 20.9, 21.2, 21.8, 22.2, 23.1, 23.5, and 27.8. For example, a contemplated crystalline form has a powder X-ray diffraction pattern shown in FIG. 19. In one embodiment, the powder X-ray diffraction pattern of the crystalline form was obtained using Cu Kα radiation.

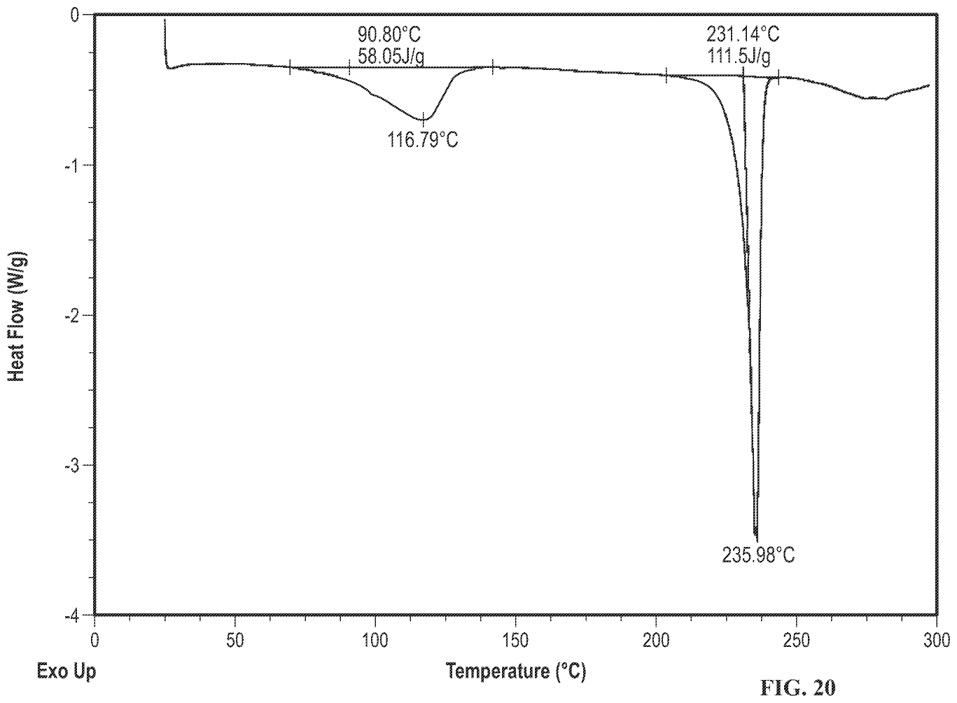

The contemplated crystalline Form 5-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt, may be characterized by a differential scanning calorimetry (DSC) profile showing a characteristic endotherm with an onset of about 91° C. and a peak of about 117° C. (enthalpy 58.0 J/g), and a characteristic endotherm with an onset of about 231° C. and a peak of about 236° C. (enthalpy 111.5 J/g). Form 5-1, for example, may be characterized by the differential scanning calorimetry profile shown in FIG. 20.

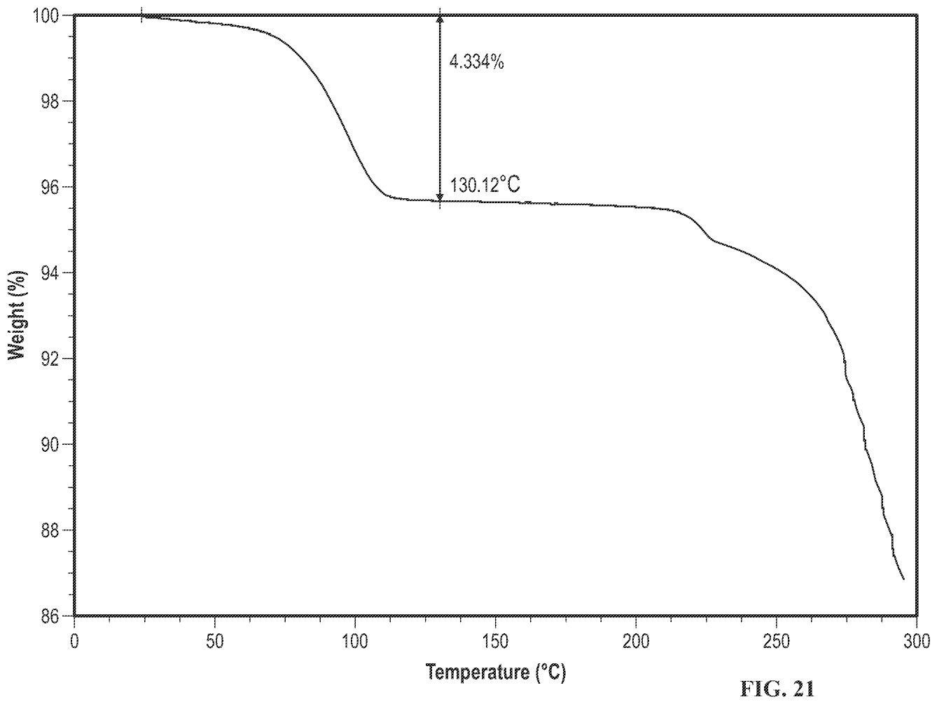

The contemplated crystalline Form 5-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt, may be characterized by a thermogravimetric analysis (TGA) profile showing a mass loss of about 4.33 wt. % between about 20° C. to about 130° C. Form 5-1, for example, may be characterized by the thermogravimetric analysis profile shown in FIG. 21. In certain embodiments, the contemplated crystalline Form 5-1 is a methanol solvate form (⅔ mol).

In another embodiment, disclosed herein is a different crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt, characterized by a powder X-ray diffraction pattern having a characteristic peak in degrees 2θ at about 16.1 (referred to herein as “Form 5-2”).

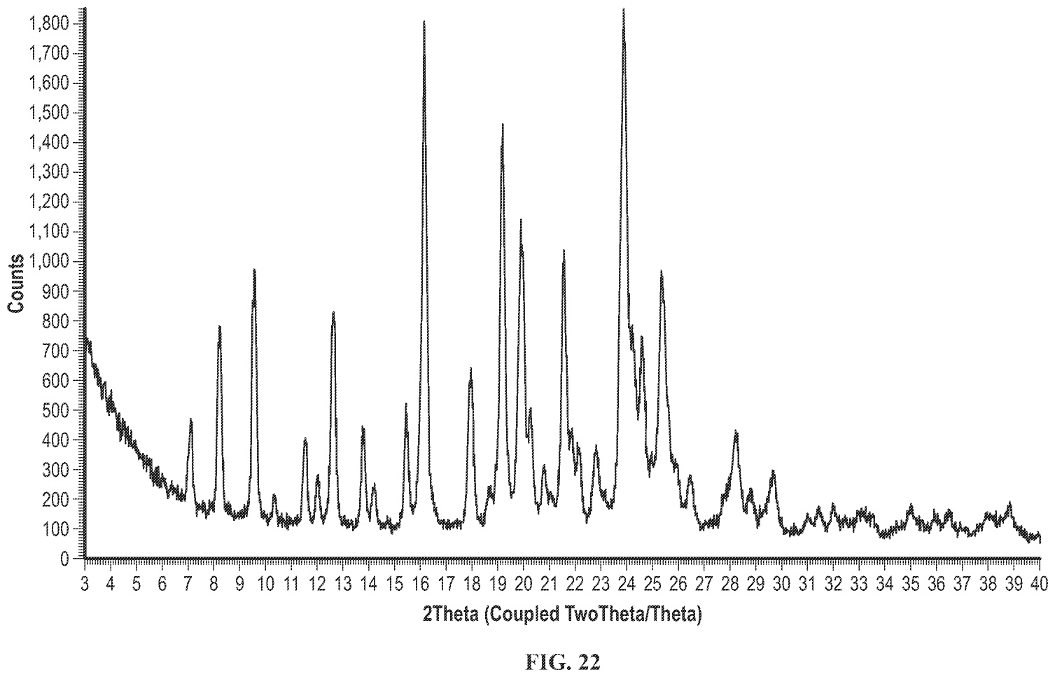

In one embodiment, the crystalline Form 5-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropane carboxamide, sulfuric acid salt, is characterized by a powder X-ray diffraction pattern that has a characteristic peak in degrees 2θ at about 8.2, is characterized by a powder X-ray diffraction pattern that has a characteristic peak in degrees 2θ at about 9.6, is characterized by a powder X-ray diffraction pattern that has a characteristic peak in degrees 2θ at about 12.6, is characterized by a powder X-ray diffraction pattern that has a characteristic peak in degrees 2θ at about 15.4, is characterized by a powder X-ray diffraction pattern that has a characteristic peak in degrees 2θ at about 16.1, is characterized by a powder X-ray diffraction pattern that has a characteristic peak in degrees 2θ at about 18.0, is characterized by a powder X-ray diffraction pattern that has a characteristic peak in degrees 2θ at about 19.2, is characterized by a powder X-ray diffraction pattern that has a characteristic peak in degrees 2θ at about 19.9, is characterized by a powder X-ray diffraction pattern that has a characteristic peak in degrees 2θ at about 20.3, is characterized by a powder X-ray diffraction pattern that has a characteristic peak in degrees 2θ at about 21.6, is characterized by a powder X-ray diffraction pattern that has a characteristic peak in degrees 2θ at about 23.9, is characterized by a powder X-ray diffraction pattern that has a characteristic peak in degrees 2θ at about 24.2, is characterized by a powder X-ray diffraction pattern that has a characteristic peak in degrees 2θ at about 24.6, is characterized by a powder X-ray diffraction pattern that has a characteristic peak in degrees 2θ at about 25.4, and/or is characterized by a powder X-ray diffraction pattern that has a characteristic peak in degrees 2θ at about 28.2. In another embodiment, crystalline Form 5-2 is characterized by a powder X-ray diffraction pattern having at least one or more characteristic peaks in degrees 2θ at about 16.1, 19.2, and 23.9. In a further embodiment, crystalline Form 5-2 is characterized by a powder X-ray diffraction pattern having at least one or more characteristic peaks in degrees 2θ at about 9.6, 16.1, 19.2, 19.9, 21.6, and 23.9. In yet another embodiment, crystalline Form 5-2 is characterized by a powder X-ray diffraction pattern having at least one or more characteristic peaks in degrees 2θ at about 9.6, 12.6, 16.1, 19.2, 19.9, 21.6, 23.9, 24.2, and 25.4. In another embodiment, crystalline Form 5-2 is characterized by a powder X-ray diffraction pattern having at least one or more characteristic peaks in degrees 2θ at about 8.2, 9.6, 12.6, 16.1, 18.0, 19.2, 19.9, 21.6, 23.9, 24.2, 24.6, and 25.4. In another embodiment, crystalline Form 5-2 is characterized by a powder X-ray diffraction pattern having at least one or more characteristic peaks in degrees 2θ at about 8.2, 9.6, 12.6, 15.4, 16.1, 18.0, 19.2, 19.9, 20.3, 21.6, 23.9, 24.2, 24.6, 25.4, and 28.2. For example, a contemplated crystalline form has a powder X-ray diffraction pattern shown in FIG. 22. In one embodiment, the powder X-ray diffraction pattern of the crystalline form was obtained using Cu Kat radiation.

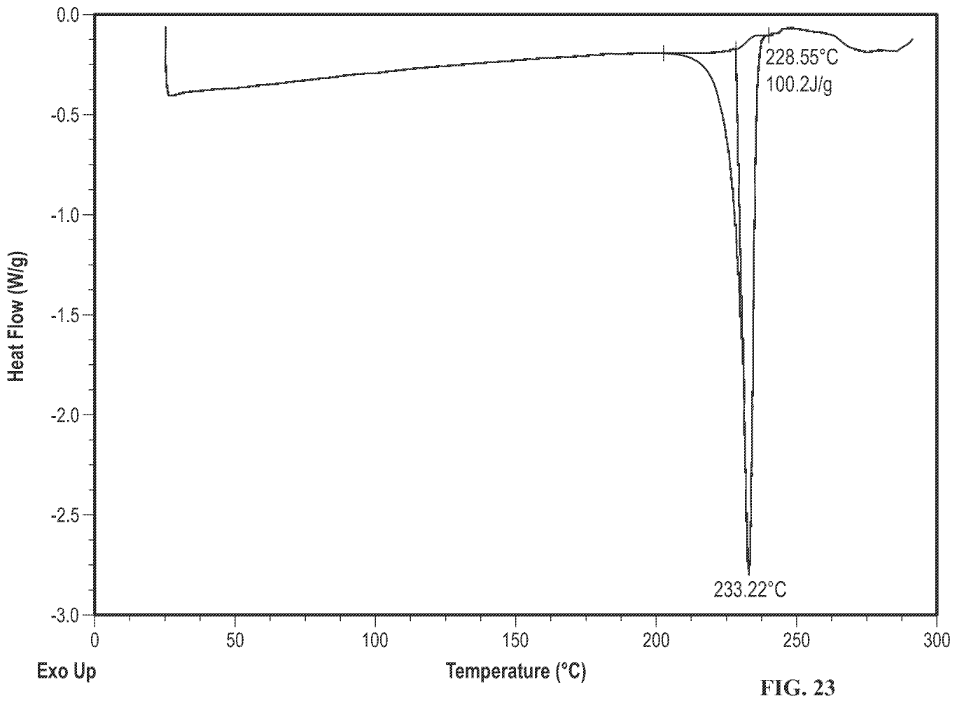

The contemplated crystalline Form 5-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt, may be characterized by a differential scanning calorimetry (DSC) profile showing a characteristic endotherm with an onset of about 229° C. and a peak of about 233° C. (enthalpy 100.2 J/g). Form 5-2, for example, may be characterized by the differential scanning calorimetry profile shown in FIG. 23.

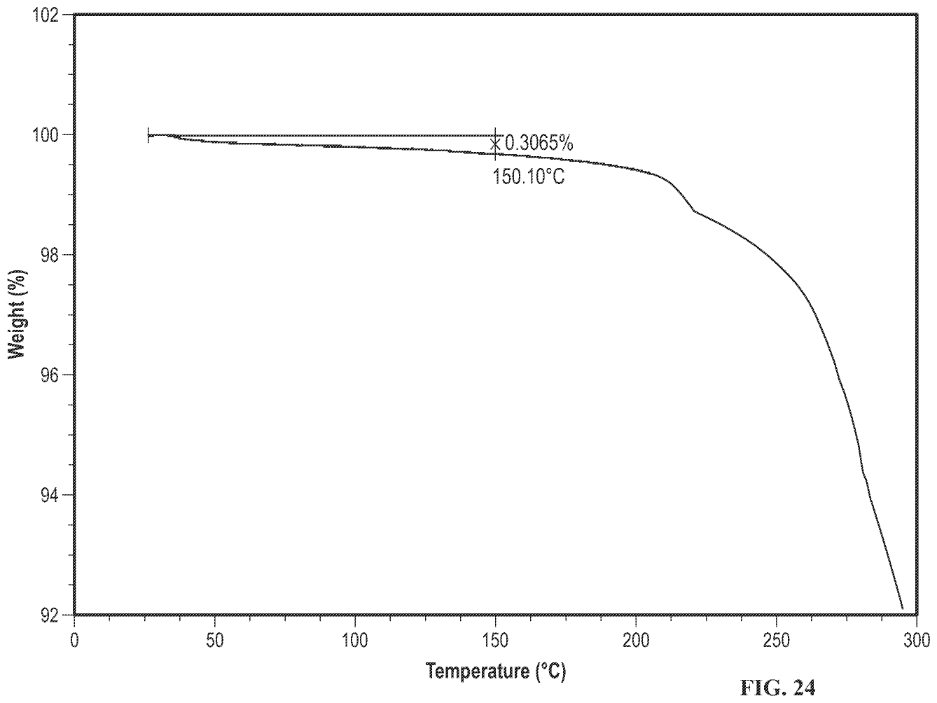

The contemplated crystalline Form 5-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt, may be characterized by a thermogravimetric analysis (TGA) profile showing a mass loss of about 0.3 wt. % between about 20° C. to about 150° C. Form 5-2, for example, may be characterized by the thermogravimetric analysis profile shown in FIG. 24. In some embodiments, crystalline Form 5-2 may be characterized by a dynamic vapor sorption (DVS) profile showing a reversable total mass change of about 1.4 wt. % between about 0% to about 90% relative humidity (RH) at 25° C. In certain embodiments, the contemplated crystalline Form 5-2 is an anhydrous crystalline form.

In another embodiment, disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, p-toluenesulfonic acid salt, characterized by a powder X-ray diffraction pattern having a characteristic peak in degrees 2θ at about 9.8 (referred to herein as “Form 6”).

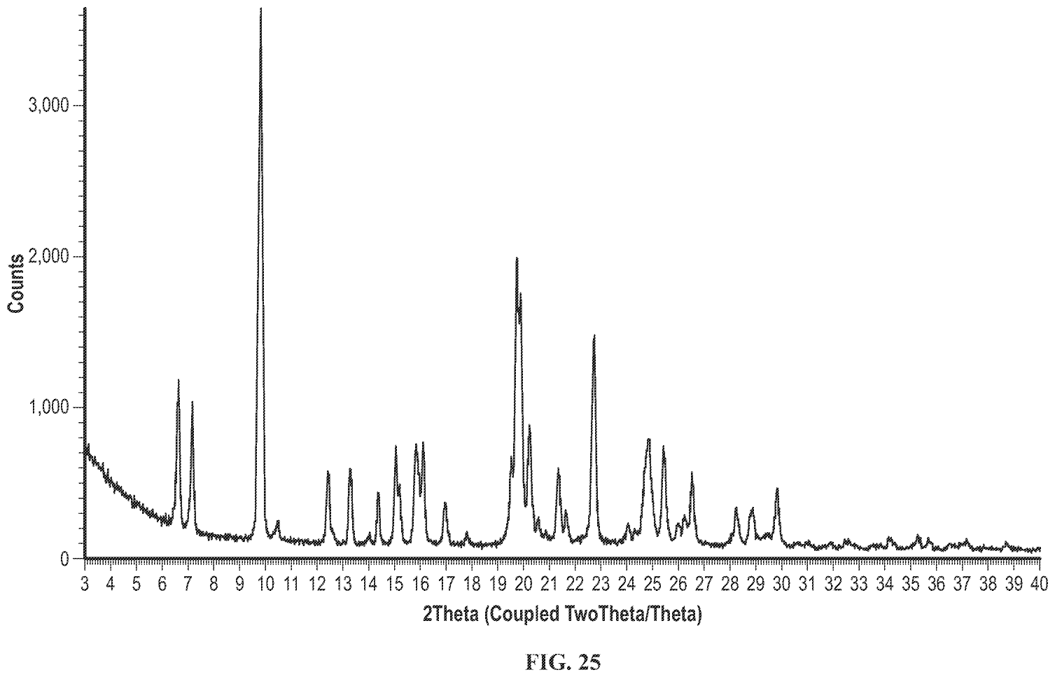

In one embodiment, the crystalline Form 6 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropane carboxamide, p-toluenesulfonic acid salt, is characterized by a powder X-ray diffraction pattern that has a characteristic peak in degrees 2θ at about 6.6, is characterized by a powder X-ray diffraction pattern that has a characteristic peak in degrees 2θ at about 7.2, is characterized by a powder X-ray diffraction pattern that has a characteristic peak in degrees 2θ at about 9.8, is characterized by a powder X-ray diffraction pattern that has a characteristic peak in degrees 2θ at about 13.3, is characterized by a powder X-ray diffraction pattern that has a characteristic peak in degrees 2θ at about 15.1, is characterized by a powder X-ray diffraction pattern that has a characteristic peak in degrees 2θ at about 15.8, is characterized by a powder X-ray diffraction pattern that has a characteristic peak in degrees 2θ at about 16.1, is characterized by a powder X-ray diffraction pattern that has a characteristic peak in degrees 2θ at about 19.5, is characterized by a powder X-ray diffraction pattern that has a characteristic peak in degrees 2θ at about 19.8, is characterized by a powder X-ray diffraction pattern that has a characteristic peak in degrees 2θ at about 20.2, is characterized by a powder X-ray diffraction pattern that has a characteristic peak in degrees 2θ at about 21.4, is characterized by a powder X-ray diffraction pattern that has a characteristic peak in degrees 2θ at about 22.7, is characterized by a powder X-ray diffraction pattern that has a characteristic peak in degrees 2θ at about 24.9, is characterized by a powder X-ray diffraction pattern that has a characteristic peak in degrees 2θ at about 25.4, and/or is characterized by a powder X-ray diffraction pattern that has a characteristic peak in degrees 2θ at about 26.5. In another embodiment, crystalline Form 6 is characterized by a powder X-ray diffraction pattern having at least one or more characteristic peaks in degrees 2θ at about 9.8, 19.8, and 22.7. In a further embodiment, crystalline Form 6 is characterized by a powder X-ray diffraction pattern having at least one or more characteristic peaks in degrees 2θ at about 6.6, 7.2, 9.8, 19.8, 20.2, and 22.7. In yet another embodiment, crystalline Form 6 is characterized by a powder X-ray diffraction pattern having at least one or more characteristic peaks in degrees 2θ at about 6.6, 7.2, 9.8, 16.1, 19.8, 20.2, 22.7, 24.9, and 25.4. In another embodiment, crystalline Form 6 is characterized by a powder X-ray diffraction pattern having at least one or more characteristic peaks in degrees 2θ at about 6.6, 7.2, 9.8, 15.1, 15.8, 16.1, 19.5, 19.8, 20.2, 22.7, 24.9, and 25.4. In another embodiment, crystalline Form 6 is characterized by a powder X-ray diffraction pattern having at least one or more characteristic peaks in degrees 2θ at about 6.6, 7.2, 9.8, 13.3, 15.1, 15.8, 16.1, 19.5, 19.8, 20.2, 21.4, 22.7, 24.9, 25.4, and 26.5. For example, a contemplated crystalline form has a powder X-ray diffraction pattern shown in FIG. 25. In one embodiment, the powder X-ray diffraction pattern of the crystalline form was obtained using Cu Kat radiation.

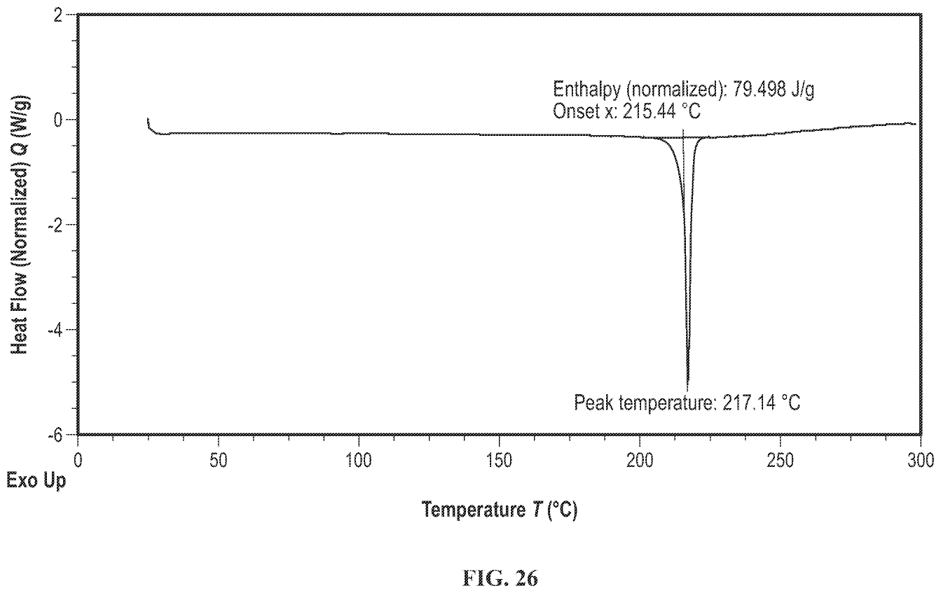

The contemplated crystalline Form 6 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, p-toluenesulfonic acid salt, may be characterized by a differential scanning calorimetry (DSC) profile showing a characteristic endotherm with an onset of about 215° C. and a peak of about 217° C. (enthalpy 79.5 J/g). Form 6, for example, may be characterized by the differential scanning calorimetry profile shown in FIG. 26.

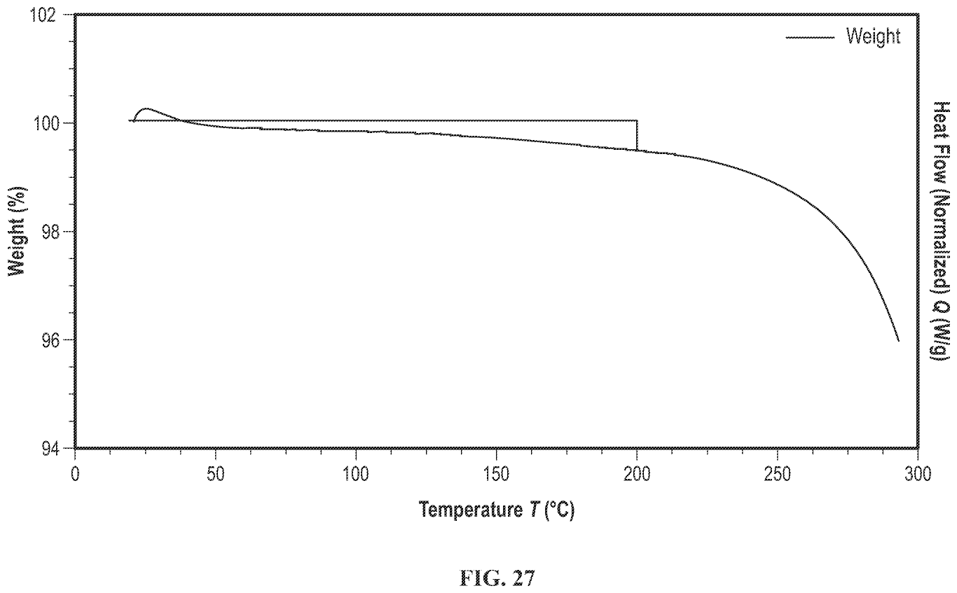

The contemplated crystalline Form 6 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, p-toluenesulfonic acid salt, may be characterized by a thermogravimetric analysis (TGA) profile showing a mass loss of about 0.6 wt. % between about 20° C. to about 200° C. Form 6, for example, may be characterized by the thermogravimetric analysis profile shown in FIG. 27. In some embodiments, crystalline Form 6 may be characterized by a dynamic vapor sorption (DVS) profile showing a reversable total mass change of about 0.7 wt. % between about 0% to about 90% relative humidity (RH) at 25° C. In certain embodiments, the contemplated crystalline Form 6 is an anhydrous crystalline form.

In another embodiment, disclosed herein is a crystalline form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, bis-hydrochloride salt, characterized by a powder X-ray diffraction pattern having a characteristic peak in degrees 2θ at about 9.4 (referred to herein as “Form 7”).

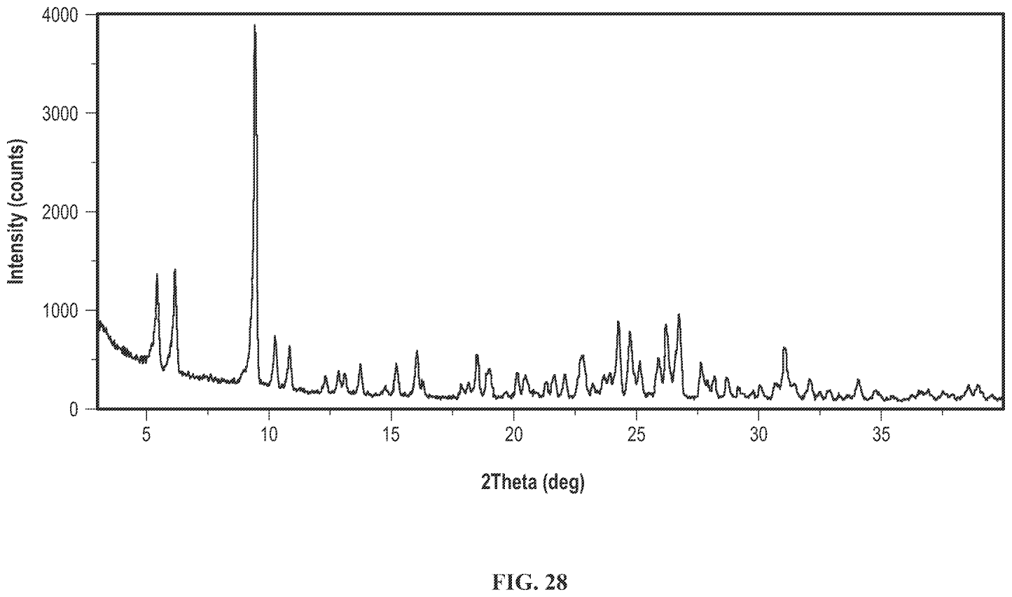

In one embodiment, the crystalline Form 7 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropane carboxamide, bis-hydrochloride salt, is characterized by a powder X-ray diffraction pattern that has a characteristic peak in degrees 2θ at about 5.4, is characterized by a powder X-ray diffraction pattern that has a characteristic peak in degrees 2θ at about 6.2, is characterized by a powder X-ray diffraction pattern that has a characteristic peak in degrees 2θ at about 9.4, is characterized by a powder X-ray diffraction pattern that has a characteristic peak in degrees 2θ at about 10.3, is characterized by a powder X-ray diffraction pattern that has a characteristic peak in degrees 2θ at about 10.8, is characterized by a powder X-ray diffraction pattern that has a characteristic peak in degrees 2θ at about 16.0, is characterized by a powder X-ray diffraction pattern that has a characteristic peak in degrees 2θ at about 18.5, is characterized by a powder X-ray diffraction pattern that has a characteristic peak in degrees 2θ at about 24.3, is characterized by a powder X-ray diffraction pattern that has a characteristic peak in degrees 2θ at about 24.7, is characterized by a powder X-ray diffraction pattern that has a characteristic peak in degrees 2θ at about 26.2, is characterized by a powder X-ray diffraction pattern that has a characteristic peak in degrees 2θ at about 26.7, and/or is characterized by a powder X-ray diffraction pattern that has a characteristic peak in degrees 2θ at about 31.0. In another embodiment, crystalline Form 7 is characterized by a powder X-ray diffraction pattern having at least one or more characteristic peaks in degrees 2θ at about 5.4, 6.2, and 9.4. In a further embodiment, crystalline Form 7 is characterized by a powder X-ray diffraction pattern having at least one or more characteristic peaks in degrees 2θ at about 5.4, 6.2, 9.4, 24.3, 26.2, and 26.7. In yet another embodiment, crystalline Form 6 is characterized by a powder X-ray diffraction pattern having at least one or more characteristic peaks in degrees 2θ at about 5.4, 6.2, 9.4, 10.3, 24.3, 24.7, 26.2, 26.7, and 31.0. In another embodiment, crystalline Form 7 is characterized by a powder X-ray diffraction pattern having at least one or more characteristic peaks in degrees 2θ at about 5.4, 6.2, 9.4, 10.3, 10.8, 16.0, 18.5, 24.3, 24.7, 26.2, 26.7, and 31.0. For example, a contemplated crystalline form has a powder X-ray diffraction pattern shown in FIG. 28. In one embodiment, the powder X-ray diffraction pattern of the crystalline form was obtained using Cu Kα radiation.

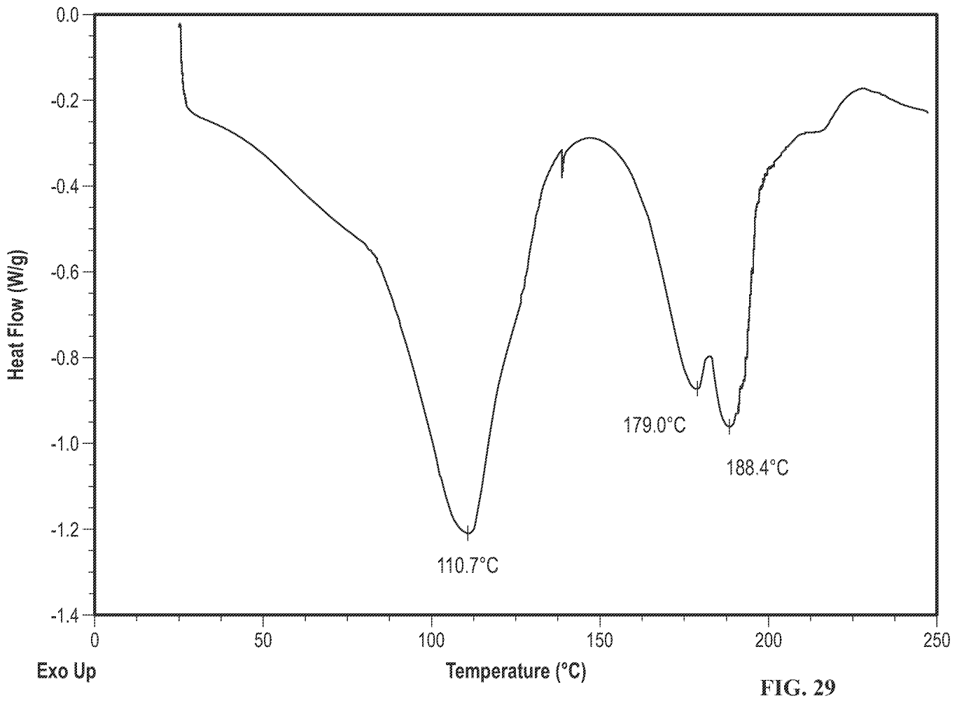

The contemplated crystalline Form 7 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, bis-hydrochloride salt, may be characterized by a differential scanning calorimetry (DSC) profile showing a characteristic endotherm with a peak of about 111° C., a characteristic endotherm with a peak of about 179° C., and a characteristic endotherm with a peak of about 188° C. Form 7, for example, may be characterized by the differential scanning calorimetry profile shown in FIG. 29.

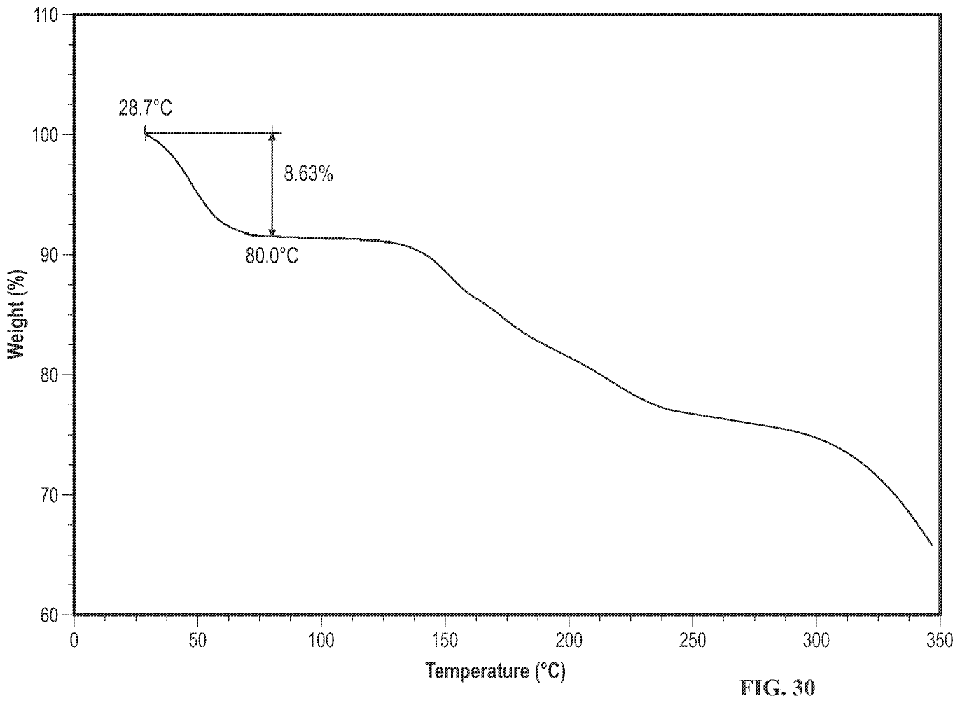

The contemplated crystalline Form 7 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, bis-hydrochloride salt, may be characterized by a thermogravimetric analysis (TGA) profile showing a mass loss of about 8.63 wt. % between about 29° C. to about 80° C. Form 7, for example, may be characterized by the thermogravimetric analysis profile shown in FIG. 30.

In a further embodiment, a pharmaceutical composition comprising a disclosed crystalline salt form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide and a pharmaceutically acceptable excipient is disclosed herein. In another embodiment, a pharmaceutical composition formed from a disclosed crystalline salt form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide disclosed herein. In some embodiments, a disclosed pharmaceutical composition may be a formulation for oral administration.

In an embodiment, a drug substance comprising at least a detectable amount of a disclosed crystalline salt form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide is disclosed herein. In another embodiment, a drug substance comprising a substantially pure crystalline salt form of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide is disclosed herein.

### Compositions

Another aspect of the disclosure provides pharmaceutical compositions comprising crystalline compounds as disclosed herein formulated together with a pharmaceutically acceptable excipient. In particular, the present disclosure provides pharmaceutical compositions comprising crystalline compounds as disclosed herein formulated together with one or more pharmaceutically acceptable excipients. These formulations include those suitable for oral, topical (e.g., transdermal), buccal, ocular, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral, subcutaneous or intravenous administration.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic excipient or excipient suitable for external, enteral or parenteral applications. The active ingredient may be compounded, for example, with the usual non-toxic, pharmaceutically acceptable excipients for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical excipient, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable excipients, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, nano-suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or excipients comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable excipient, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable excipients and stabilizers. The excipients and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous excipients which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants. For example, crystalline forms provided herein may be milled to obtain a particular particle size, and in at least some embodiments, such crystalline forms may remain substantially stable upon milling.

Amounts of a crystalline compound as described herein in a formulation may vary according to factors such as the disease state, age, sex, and weight of the individual. Dosage regimens can be adjusted to provide the optimum therapeutic response. For example, a single bolus can be administered, several divided doses may be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active crystalline compound calculated to produce the desired therapeutic effect in association with the required pharmaceutical carrier.

The specification for the dosage unit forms of the disclosure are dictated by and directly dependent on (a) the unique characteristics of the crystalline compound selected and the particular therapeutic effect to be achieved, and (b) the limitations inherent in the art of compounding such an active crystalline compound for the treatment of sensitivity in individuals.

Disclosed compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is suitable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin.

A disclosed crystalline compound can be administered in a time release formulation, for example in a composition which includes a slow release polymer. The crystalline compound can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

In accordance with an alternative aspect of the disclosure, a disclosed crystalline compound can be formulated with one or more additional compounds that enhance the solubility of the compound.

### Methods

The crystalline forms disclosed herein are useful for the inhibition of kinase activity of one or more enzymes. In some embodiments the kinase inhibited by the crystalline forms and methods is TYK2.

Provided herein are crystalline salt forms of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide that are inhibitors of TYK2 and are therefore useful for treating one or more disorders associated with activity of TYK2 or mutants thereof.

Provided herein are methods for treating a disease or disorder in a patient in need thereof, wherein the disease or disorder is an autoimmune disorders, inflammatory disorders, proliferative disorders, endocrine disorders, neurological disorders, or disorders associated with transplantation, comprising administering to the patient an effective amount of a crystalline salt form described herein, or a pharmaceutical composition comprising an effective amount of a disclosed crystalline salt form.

In some embodiments, the disease or disorder is an autoimmune disorder. In some embodiments the disease or disorder is selected from type 1 diabetes, systemic lupus erythematosus, multiple sclerosis, psoriasis, Behçet's disease, POEMS syndrome, Crohn's disease, ulcerative colitis, and inflammatory bowel disease.

In some embodiments, the disease or disorder is an inflammatory disorder. In some embodiments, the inflammatory disorder is rheumatoid arthritis, asthma, chronic obstructive pulmonary disease, psoriasis, hepatomegaly, Crohn's disease, ulcerative colitis, inflammatory bowel disease.

In some embodiments, the disease or disorder is a proliferative disorder. In some embodiments, the proliferative disorder is cancer. In some embodiments, the disease or disorder is a proliferative disorder. In some embodiments, the proliferative disorder is a hematological cancer. In some embodiments the proliferative disorder is a leukemia. In some embodiments, the leukemia is a T-cell leukemia. In some embodiments the T-cell leukemia is T-cell acute lymphoblastic leukemia (T-ALL). In some embodiments the proliferative disorder is polycythemia vera, myelofibrosis, essential or thrombocytosis.

In some embodiments, the disease or disorder is an endocrine disorder. In some embodiments, the endocrine disorder is polycystic ovary syndrome, Crouzon's syndrome, or type 1 diabetes.

In some embodiments, the disease or disorder is a neurological disorder. In some embodiments, the neurological disorder is Alzheimer's disease.

In some embodiments the proliferative disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and R1027H.

In some embodiments, the disease or disorder is associated with transplantation. In some embodiments the disease or disorder associated with transplantation is transplant rejection, or graft versus host disease.

In some embodiments the disease or disorder is associated with type I interferon, IL-10, IL-12, or IL-23 signaling. In some embodiments the disease or disorder is associated with type I interferon signaling. In some embodiments the disease or disorder is associated with IL-10 signaling. In some embodiments the disorder is associated with IL-12 signaling. In some embodiments the disease or disorder is associated with IL-23 signaling.

Provided herein are methods for treating an inflammatory or allergic condition of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Provided herein are methods for treating other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease is acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), or osteoarthritis.

In some embodiments the inflammatory disease is a Th1 or Th17 mediated disease. In some embodiments the Th17 mediated disease is selected from Systemic lupus erythematosus, Multiple sclerosis, and inflammatory bowel disease (including Crohn's disease or ulcerative colitis).

In some embodiments the inflammatory disease is Sjogren's syndrome, allergic disorders, osteoarthritis, conditions of the eye such as ocular allergy, conjunctivitis, keratoconjunctivitis sicca, vernal conjunctivitis, or diseases affecting the nose such as allergic rhinitis.

For example, disclosed herein is a method of inhibiting a TYK2 enzyme in a patient or biological sample, comprising contacting said patient or biological sample with a therapeutically effective amount of a crystalline salt form described herein, or a pharmaceutical composition comprising an effective amount of a disclosed crystalline salt form.

Also disclosed herein is method of inhibiting TYK2 activity in a patient in need thereof, comprising administering to the patient a therapeutically effective amount of a crystalline salt form described herein, or a pharmaceutical composition comprising an effective amount of a disclosed crystalline salt form. In some embodiments, inhibiting TYK2 activity is associated with treating a disease or disorder selected from the group consisting of, e.g., Crohn's disease, rheumatoid arthritis, psoriasis, systemic lupus erythematosus, ulcerative colitis, psoriatic arthritis, and systemic sclerosis.

Further disclosed herein is a TYK2-mediated disorder in a patient in need thereof, comprising administering to the patient a therapeutically effective amount of a crystalline salt form described herein, or a pharmaceutical composition comprising an effective amount of a disclosed crystalline salt form. In some embodiments, a contemplated TYK2-mediated disorder may be, for example, an autoimmune disorder, an inflammatory disorder, a proliferative disorder, an endocrine disorder, a neurological disorder, or a disorder associated with transplantation. In other embodiments, a contemplated disorder is associated with type I interferon, IL-10, IL-12, or IL-23 signalling.

For example, provided herein is a method of treating one or more of: Crohn's disease, rheumatoid arthritis, psoriasis, systemic lupus erythematosus, ulcerative colitis, psoriatic arthritis, and systemic sclerosis in a patient in need thereof, comprising administering to the patient an effective amount of a crystalline salt form described herein, or a pharmaceutical composition comprising an effective amount of a disclosed crystalline salt form.

In particular, in certain embodiments, the disclosure provides a method of treating the above medical indications comprising administering to a patient in need thereof an effective amount of a crystalline salt form described herein, or a pharmaceutical composition comprising an effective amount of a disclosed crystalline salt form. In certain other embodiments, the disclosure provides a method of treating the above medical conditions in a patient in need thereof, comprising orally, subcutaneously, or intravenously administering to the patient a composition comprising a crystalline salt form described herein, or a pharmaceutical composition comprising an effective amount of a disclosed crystalline salt form.

The crystalline compounds disclosed herein can be used as a medicament or pharmaceutically acceptable composition, e.g., in the form of pharmaceutical preparations for oral, enteral, parenteral, or topical administration, and the contemplated methods disclosed herein may include administering orally, enterally, parenterally, or topically a disclosed crystalline compound, or a composition comprising or formed from such a disclosed crystalline compound. For example, a disclosed crystalline salt form may be capable of controlling one or more pharmacokinetic properties (e.g., a longer or shorter release profile) when administered by a certain route (e.g., oral) or in a certain formulation, as compared to a different route (e.g., subcutaneous) or other formulation e.g., a formulation having the amorphous form. In one embodiment, a disclosed crystalline salt form may afford substantial reproducibility from one formulation to another.

### EXAMPLES

The compounds and crystalline salt forms described herein can be prepared in a number of ways based on the teachings contained herein and synthetic procedures known in the art. The following non-limiting examples illustrate the disclosure.

X-ray powder diffraction was performed using a Bruker D8 Advance X-ray diffractometer equipped with a LynxEye detector. Samples were scanned from 3 to 40° 2θ, at a step size 0.02° 2θ. The radiation was Cu/K-Alpha1 (λ=1.5406 Å). The tube voltage and current were 40 kV and 40 mA, respectively.

Differential scanning calorimetry (DSC) was performed using a Discovery DSC 250 (TA Instruments, US) calorimeter. The sample was placed into an aluminum pin-hole hermetic pan and the weight was accurately recorded. The sample was heated at a rate of 10° C./min from 25° C. to the final temperature.

Thermal gravimetric analysis (TGA) was performed on a TGA Q500 or Discovery TGA 55 (TA Instruments, US). The sample was placed into an open tared aluminum pan, automatically weighed, and inserted into the TGA furnace. The sample was heated at a rate of 10° C./min from ambient temperature to the final temperature.

Dynamic vapor sorption analysis (DVS) was performed on a DVS Intrinsic PLUS (SMS, UK). The sample was placed into a tared sample chamber and automatically weighed. The sample was dried at 40° C. until the dm/dt was less than 0.002% and cooled to 25° C. The instrument parameters were as follows: step time (min)=60 min; sample temperature=25 C; cycle=full cycle; adsorption (% RH)=0, 10, 20, 30, 40, 50, 60, 70, 80, 90; desorption (% RH)=80, 70, 60, 50, 40, 30, 20, 10, 0; saved data rate=5 sec; total flow rate=200 sccm; post experimental flow rate=200 sccm.

Polarized Light Microscopy (PLM) was performed using a Polarizing Microscope ECLIPSE LV100POL (Nikon, JPN).

### Example 1

Crystalline Form 1-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (30-45 mg) was added into MeOH (9 V). After addition of 1.1 eq. methanesulfonic acid, precipitation occurred after addition of 19 volumes of ethyl acetate. The mixture was concentrated to about 3 volumes at room temperature and stirred. The solids were collected and dried under vacuum. XRPD analysis indicated that the dried material was crystalline with a pattern consistent with Form 1-1.

Crystalline Form 1-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, was also prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (30-45 mg) was added into MeOH (5 V) with heating. After addition of 1.1 eq. methanesulfonic acid, precipitation occurred after addition of ethyl acetate (14 V). The mixture was cooled and stirred at room temperature, then an additional 10 volumes of ethyl acetate were added. The solids were collected and dried under vacuum. XRPD analysis indicated that the dried material was crystalline with a pattern consistent with Form 1-1.

The XRPD pattern of crystalline Form 1-1 is shown in FIG. 1. Characteristic peaks include one or more of the peaks shown in Table 1.

TABLE 1

| Angle | d Value | Peak Height | Rel. Intensity |
|---|---|---|---|
| (deg 2 theta) | (Å) | (counts) | (%) |
| 39.383 | 2.28605 | 148.248 | 4.0 |
| 38.869 | 2.31509 | 112.608 | 2.1 |
| 38.449 | 2.33944 | 104.745 | 1.7 |
| 37.622 | 2.38893 | 151.397 | 4.2 |
| 36.025 | 2.49104 | 127.729 | 3.0 |
| 35.304 | 2.54028 | 136.058 | 3.4 |
| 34.735 | 2.58054 | 302.748 | 12.1 |
| 33.953 | 2.63822 | 152.912 | 4.0 |
| 32.996 | 2.71250 | 319.938 | 13.0 |
| 32.780 | 2.72984 | 171.572 | 4.9 |
| 32.404 | 2.76067 | 107.459 | 1.4 |
| 31.662 | 2.82364 | 172.277 | 4.5 |
| 31.460 | 2.84138 | 171.842 | 4.4 |
| 30.868 | 2.89447 | 235.681 | 7.5 |
| 30.209 | 2.95608 | 207.917 | 5.9 |
| 29.659 | 3.00962 | 272.3 | 9.4 |
| 28.440 | 3.13587 | 164.529 | 3.6 |
| 28.179 | 3.16432 | 197.614 | 5.3 |
| 27.804 | 3.20614 | 206.616 | 5.7 |
| 26.864 | 3.31615 | 956.151 | 45.3 |
| 26.420 | 3.37081 | 451.992 | 17.9 |
| 25.835 | 3.44574 | 234.534 | 5.9 |
| 25.544 | 3.48442 | 634.119 | 27.2 |
| 25.282 | 3.51988 | 475.696 | 18.6 |
| 25.041 | 3.55325 | 520.255 | 21.0 |
| 24.566 | 3.62083 | 214.09 | 4.7 |
| 24.298 | 3.66015 | 998.368 | 46.7 |
| 23.952 | 3.71227 | 1990.65 | 100.0 |
| 23.669 | 3.75607 | 962.971 | 45.2 |
| 23.447 | 3.79109 | 338.975 | 12.0 |
| 22.633 | 3.92555 | 262.692 | 8.4 |
| 21.960 | 4.04421 | 211.584 | 5.6 |
| 21.519 | 4.12615 | 176.683 | 3.4 |
| 21.237 | 4.18028 | 189.958 | 3.9 |
| 20.927 | 4.24157 | 750.117 | 33.7 |
| 20.755 | 4.27620 | 675.16 | 29.7 |
| 20.507 | 4.32752 | 1063.25 | 50.4 |
| 19.988 | 4.43861 | 162.751 | 2.2 |
| 19.715 | 4.49948 | 385.9 | 14.1 |
| 19.378 | 4.57701 | 643.671 | 28.0 |
| 19.095 | 4.64423 | 1322.41 | 64.4 |
| 18.286 | 4.84784 | 608.478 | 26.5 |
| 17.100 | 5.18113 | 1703.69 | 85.4 |
| 16.671 | 5.31366 | 264.688 | 8.5 |
| 16.007 | 5.53255 | 349.091 | 13.1 |
| 15.524 | 5.70362 | 963.986 | 46.0 |
| 14.897 | 5.94200 | 318.368 | 11.7 |
| 13.398 | 6.60347 | 384.162 | 14.8 |
| 13.003 | 6.80298 | 142.604 | 1.6 |
| 12.658 | 6.98789 | 1649.7 | 82.1 |
| 12.005 | 7.36611 | 355.592 | 12.8 |
| 11.315 | 7.81404 | 350.105 | 12.4 |
| 10.400 | 8.49931 | 189.078 | 3.4 |
| 9.485 | 9.31735 | 1208.21 | 57.4 |
| 7.787 | 11.34406 | 997.678 | 44.7 |
| 6.909 | 12.78336 | 974.416 | 42.7 |

FIG. 2 depicts the differential scanning calorimetry (DSC) profile of crystalline Form 1-1. As shown in FIG. 2, crystalline Form 1-1 shows a characteristic endotherm with an onset of about 200° C. and a peak of about 202° C. (enthalpy 98.7 J/g).

Crystalline Form 1-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide methanesulfonic acid salt, displayed a thermogravimetric analysis (TGA) profile showing a mass loss of about 0.27 wt. % between about 20° C. to about 180° C. (FIG. 3). Crystalline Form 1-1 displayed a dynamic vapor sorption (DVS) profile showing a reversable total mass change of about 0.6 wt. % between about 0% to about 80% relative humidity (RH) at 25° C., and a reversable total mass change of about 1.3 wt. % between about 0% to about 90% relative humidity (RH) at 25° C. No form change was observed after DVS analysis.

Crystalline Form 1-1 displayed a solubility of >4.1 mg/mL in SGF, a solubility of 0.02-0.03 mg/mL in FeSSIF, a solubility of 2.06-0.93 mg/mL in water, and a solubility of 0.001-0.002 mg/mL in FaSSIF. Crystalline Form 1-1 was observed to dissociate to the free base in FaSSIF, FeSSIF and water.

Crystalline Form 1-1 was observed to be an anhydrate and only slightly hygroscopic. Form 1-1 was observed to be chemically and physically stable at 40° C./75% RH and 60° C. in the solid form for at least 10 days.

### Example 2

Crystalline Form 1-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (30-45 mg) was added into THF (9 V). After addition of 1.1 eq. methanesulfonic acid, formation of a slurry was observed, and the solids were collected and dried under vacuum. XRPD analysis indicated that the dried material was crystalline with a pattern consistent with Form 1-2.

An XRPD pattern of crystalline Form 1-2 is shown in FIG. 4. Characteristic peaks include one or more of the peaks shown in Table 2.

TABLE 2

| Angle | d Value | Peak Height | Rel. Intensity |
|---|---|---|---|
| (deg 2 theta) | (Å) | (counts) | (%) |
| 31.936 | 2.80011 | 139.426 | 2.2 |
| 28.409 | 3.13918 | 177.662 | 2.2 |
| 26.510 | 3.35960 | 316.735 | 6.9 |
| 25.669 | 3.46770 | 196.505 | 1.7 |
| 25.153 | 3.53761 | 245.844 | 3.7 |
| 23.947 | 3.71301 | 339.797 | 7.0 |
| 22.498 | 3.94870 | 427.898 | 9.6 |
| 21.073 | 4.21247 | 348.617 | 6.4 |
| 19.973 | 4.44194 | 737.35 | 23.0 |
| 18.188 | 4.87354 | 362.15 | 8.9 |
| 16.829 | 5.26400 | 190.47 | 2.5 |
| 15.566 | 5.68828 | 220.679 | 4.1 |
| 13.699 | 6.45895 | 327.629 | 8.5 |
| 12.525 | 7.06179 | 142.972 | 1.0 |
| 10.374 | 8.52025 | 322.809 | 6.2 |
| 9.916 | 8.91319 | 2641.11 | 100.0 |
| 9.224 | 9.57991 | 402.375 | 8.2 |
| 8.340 | 10.59312 | 325.718 | 4.5 |
| 7.530 | 11.73096 | 482.298 | 10.8 |
| 6.827 | 12.93753 | 273.026 | 2.4 |

FIG. 5 depicts the differential scanning calorimetry (DSC) profile of crystalline Form 1-2. As shown in FIG. 5, crystalline Form 1-2 shows a characteristic endotherm with a peak of about 66° C. (enthalpy 7.45 J/g), a characteristic endotherm with an onset of about 122° C. and a peak of about 132° C. (enthalpy 2.68 J/g), a characteristic endotherm with an onset of about 153° C. and a peak of about 161° C. (enthalpy 27.8 J/g), and a characteristic endotherm with an onset of about 195° C. and a peak of about 195° C. (enthalpy 3.15 J/g).

Crystalline Form 1-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, displayed a thermogravimetric analysis (TGA) profile showing a mass loss of about 0.70 wt. % between about 20° C. to about 75° C., and a mass loss of about 1.24 wt. % between about 75° C. to about 160° C. (FIG. 6). Crystalline Form 1-2 was observed to be a tetrahydrofuran solvate (0.1 mol).

### Example 3

Crystalline Form 1-3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, methanesulfonic acid salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (30-45 mg) was added into acetone/water (9:1, 14 V). After addition of 1.1 eq. methanesulfonic acid, precipitation occurred after stirring at room temperature for 2 days. The solids were collected and dried under vacuum. XRPD analysis indicated that the dried material was crystalline with a pattern consistent with Form 1-3.

The XRPD pattern of crystalline Form 1-3 is shown in FIG. 7. Characteristic peaks include one or more of the peaks shown in Table 3.

TABLE 3

| Angle | d Value | Peak Height | Rel. Intensity |
|---|---|---|---|
| (deg 2 theta) | (Å) | (counts) | (%) |
| 37.951 | 2.36897 | 110.335 | 2.5 |
| 36.568 | 2.45532 | 100.392 | 1.9 |
| 35.683 | 2.51414 | 86.0161 | 1.2 |
| 34.229 | 2.61752 | 125.574 | 2.9 |
| 33.814 | 2.64874 | 94.886 | 1.3 |
| 33.018 | 2.71071 | 108.708 | 1.8 |
| 32.503 | 2.75253 | 114.695 | 2.1 |
| 32.022 | 2.79275 | 99.6121 | 1.2 |
| 31.318 | 2.85394 | 128.848 | 2.5 |
| 30.250 | 2.95218 | 141.383 | 3.3 |
| 29.531 | 3.02240 | 136.597 | 2.9 |
| 29.084 | 3.06779 | 321.295 | 11.9 |
| 28.482 | 3.13129 | 462.792 | 18.8 |
| 28.125 | 3.17024 | 150.539 | 2.8 |
| 27.601 | 3.22925 | 206.081 | 5.6 |
| 27.253 | 3.26969 | 306.876 | 10.9 |
| 26.609 | 3.34728 | 175.756 | 4.5 |
| 26.184 | 3.40060 | 187.47 | 4.9 |
| 25.729 | 3.45976 | 137.593 | 1.8 |
| 24.905 | 3.57232 | 179.817 | 3.1 |
| 24.246 | 3.66797 | 456.413 | 16.9 |
| 23.952 | 3.71229 | 423.189 | 15.1 |
| 23.284 | 3.81715 | 853.172 | 37.0 |
| 23.000 | 3.86371 | 350.673 | 11.5 |
| 22.648 | 3.92292 | 364.835 | 12.4 |
| 21.953 | 4.04558 | 285.327 | 8.5 |
| 21.473 | 4.13493 | 363.323 | 12.5 |
| 21.036 | 4.21986 | 2079.04 | 100.0 |
| 20.595 | 4.30913 | 499.662 | 19.9 |
| 20.188 | 4.39520 | 292.939 | 9.7 |
| 19.682 | 4.50688 | 136.644 | 1.9 |
| 19.147 | 4.63157 | 131.185 | 1.6 |
| 18.550 | 4.77929 | 239.289 | 7.1 |
| 18.063 | 4.90699 | 1234.91 | 57.8 |
| 17.473 | 5.07128 | 348.382 | 12.9 |
| 16.542 | 5.35473 | 467.291 | 18.7 |
| 16.288 | 5.43751 | 184.222 | 4.2 |
| 15.851 | 5.58665 | 184.116 | 4.1 |
| 15.582 | 5.68244 | 399.375 | 15.1 |
| 15.161 | 5.83906 | 278.068 | 9.1 |
| 14.419 | 6.13810 | 313.606 | 11.0 |
| 14.145 | 6.25638 | 138.926 | 2.1 |
| 13.094 | 6.75613 | 136.856 | 2.0 |
| 11.719 | 7.54565 | 372.921 | 13.3 |
| 11.566 | 7.64460 | 289.996 | 9.0 |
| 10.419 | 8.48330 | 650.757 | 26.5 |
| 8.994 | 9.82436 | 1160.81 | 50.8 |
| 8.076 | 10.93907 | 863.9 | 33.6 |
| 7.574 | 11.66273 | 361.393 | 6.6 |
| 7.150 | 12.35412 | 950.582 | 35.5 |

FIG. 8 depicts the differential scanning calorimetry (DSC) profile of crystalline Form 1-3. As shown in FIG. 8, crystalline Form 1-3 shows a characteristic endotherm with an onset of about 208° C. and a peak of about 209° C. (enthalpy 92.0 J/g).

Crystalline Form 1-3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide methanesulfonic acid salt, displayed a thermogravimetric analysis (TGA) profile showing no mass loss (0% wt.) between about 20° C. to about 150° C. (FIG. 9). Crystalline Form 1-3 was observed to be an anhydrate.

### Example 4

Crystalline Form 2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, fumaric acid salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (32 mg) was dissolved in DCM/MeOH (1:1, 19 V) at room temperature. Then, 1.1 eq. of 0.25 M fumaric acid/MeOH was added. Precipitation occurred immediately after adding the acid. The mixture was stirred at room temperature for 2 hours and the solids were collected by filtration and dried at 50° C. under vacuum. XRPD analysis indicated that the dried material was crystalline with a pattern consistent with Form 2.

The XRPD pattern of crystalline Form 2 is shown in FIG. 10. Characteristic peaks include one or more of the peaks shown in Table 4.

TABLE 4

| Angle | d Value | Peak Height | Rel. Intensity |
|---|---|---|---|
| (deg 2 theta) | (Å) | (counts) | (%) |
| 39.177 | 2.29760 | 108.811 | 3.2 |
| 36.735 | 2.44455 | 107.417 | 2.4 |
| 35.211 | 2.54679 | 100.514 | 2.0 |
| 33.940 | 2.63921 | 112.194 | 3.0 |
| 30.763 | 2.90407 | 174.975 | 6.0 |
| 30.301 | 2.94730 | 187.248 | 6.0 |
| 29.724 | 3.00321 | 206.547 | 6.6 |
| 29.236 | 3.05226 | 295.932 | 13.7 |
| 28.490 | 3.13040 | 198.057 | 4.2 |
| 27.569 | 3.23282 | 217.48 | 5.5 |
| 26.060 | 3.41654 | 1302.48 | 100.0 |
| 25.579 | 3.47967 | 692.699 | 45.1 |
| 25.043 | 3.55296 | 310.254 | 10.5 |
| 23.268 | 3.81980 | 1005.08 | 71.1 |
| 22.255 | 3.99141 | 901.225 | 62.3 |
| 22.032 | 4.03128 | 468.525 | 24.1 |
| 21.721 | 4.08818 | 393.519 | 17.8 |
| 21.282 | 4.17151 | 494.809 | 27.4 |
| 20.974 | 4.23203 | 522.018 | 30.3 |
| 20.493 | 4.33031 | 245.164 | 6.7 |
| 18.480 | 4.79723 | 432.873 | 26.9 |
| 18.007 | 4.92220 | 313.898 | 17.1 |
| 16.781 | 5.27900 | 293.664 | 16.0 |
| 15.830 | 5.59383 | 131.812 | 1.7 |
| 15.372 | 5.75946 | 191.671 | 6.9 |
| 14.395 | 6.14831 | 233.262 | 10.0 |
| 14.135 | 6.26060 | 233.431 | 9.8 |
| 13.453 | 6.57623 | 791.576 | 58.9 |
| 12.542 | 7.05202 | 384.722 | 22.9 |
| 11.228 | 7.87415 | 216.487 | 7.6 |
| 10.686 | 8.27221 | 439.968 | 26.5 |
| 10.342 | 8.54636 | 218.446 | 6.5 |
| 9.636 | 9.17124 | 231.234 | 6.8 |
| 9.304 | 9.49752 | 209.06 | 4.6 |
| 8.340 | 10.59270 | 509.704 | 30.1 |
| 8.004 | 11.03776 | 1105.01 | 82.4 |
| 7.502 | 11.77404 | 301.098 | 10.7 |
| 4.152 | 21.26616 | 898.175 | 38.3 |
| 3.681 | 23.98092 | 674.755 | 11.1 |

FIG. 11 depicts the differential scanning calorimetry (DSC) profile of crystalline Form 2. As shown in FIG. 11, crystalline Form 2 shows a characteristic endotherm with an onset of about 36° C. and a peak of about 57° C. (enthalpy 7.74 J/g), a characteristic endotherm with an onset of about 196 CC and a peak of about 199° C. (enthalpy 11.2 J/g), a characteristic endotherm with an onset of about 202° C. and a peak of about 205 CC (enthalpy 37.9 J/g), a characteristic endotherm with an onset of about 208 CC and a peak of about 209 CC (enthalpy 15.1 J/g), and a characteristic endotherm with an onset of about 213° C. and a peak of about 215° C. (enthalpy 21.2 J/g).

Crystalline Form 2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, fumaric acid salt, displayed a thermogravimetric analysis (TGA) profile showing a mass loss of about 0.42 wt. % between about 20° C. to about 50° C. (FIG. 12). Crystalline Form 2 was observed to be an anhydrate. Crystalline Form 2 may be characterized as having a rod-like crystalline morphology as observed by polarized light microscopy.

### Example 5

Crystalline Form 3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, hydrogen bromide salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (21 mg) was taken up in acetone/water (9:1, 14 V) at 50° C. After adding 1.1 eq. of hydrobromic acid, precipitation occurred immediately. The mixture was stirred at 50° C. for 30 min and then cooled to room temperature. The solids were collected by filtration and then analyzed after drying at 40° C. under vacuum. XRPD analysis indicated that the dried material was crystalline with a pattern consistent with Form 3.

The XRPD pattern of crystalline Form 3 is shown in FIG. 13. Characteristic peaks include one or more of the peaks shown in Table 5.

TABLE 5

| Angle | d Value | Peak Height | Rel. Intensity |
|---|---|---|---|
| (deg 2 theta) | (Å) | (counts) | (%) |
| 39.849 | 2.26042 | 169.567 | 1.4 |
| 39.438 | 2.28300 | 180.986 | 1.6 |
| 38.528 | 2.33479 | 273.908 | 4.9 |
| 37.879 | 2.37332 | 358.256 | 8.3 |
| 37.519 | 2.39524 | 196.889 | 2.1 |
| 36.732 | 2.44473 | 184.24 | 1.8 |
| 36.120 | 2.48477 | 217.555 | 2.9 |
| 35.160 | 2.55033 | 188.981 | 1.6 |
| 34.613 | 2.58937 | 257.844 | 4.0 |
| 33.588 | 2.66600 | 360.586 | 8.0 |
| 32.197 | 2.77798 | 424.904 | 10.4 |
| 31.576 | 2.83114 | 197.99 | 1.1 |
| 31.202 | 2.86424 | 238.32 | 2.4 |
| 30.800 | 2.90074 | 506.998 | 12.6 |
| 30.362 | 2.94150 | 279.174 | 3.5 |
| 29.857 | 2.99012 | 280.201 | 3.5 |
| 29.360 | 3.03957 | 729.227 | 21.0 |
| 29.107 | 3.06548 | 393.216 | 8.0 |
| 28.695 | 3.10853 | 417.442 | 9.1 |
| 27.794 | 3.20720 | 737.968 | 22.0 |
| 26.363 | 3.37792 | 866.369 | 27.3 |
| 25.685 | 3.46552 | 339.727 | 6.7 |
| 24.782 | 3.58982 | 473.128 | 11.6 |
| 24.185 | 3.67700 | 238.533 | 2.2 |
| 23.253 | 3.82226 | 961.024 | 30.2 |
| 22.899 | 3.88052 | 560.077 | 14.5 |
| 22.416 | 3.96307 | 294.328 | 4.3 |
| 21.914 | 4.05267 | 370.628 | 7.2 |
| 21.240 | 4.17974 | 877.212 | 26.7 |
| 20.858 | 4.25547 | 587.667 | 15.4 |
| 20.475 | 4.33417 | 982.231 | 30.9 |
| 19.468 | 4.55590 | 2502.99 | 90.6 |
| 17.390 | 5.09536 | 598.947 | 17.9 |
| 17.127 | 5.17307 | 493.111 | 13.9 |
| 16.603 | 5.33523 | 198.267 | 2.7 |
| 15.274 | 5.79638 | 211.474 | 3.8 |
| 13.769 | 6.42635 | 336.535 | 8.6 |
| 12.293 | 7.19458 | 208.104 | 3.3 |
| 10.554 | 8.37569 | 204.29 | 2.4 |
| 9.731 | 9.08173 | 2721.58 | 100.0 |
| 9.275 | 9.52759 | 290.653 | 5.3 |
| 8.260 | 10.69539 | 355.052 | 7.8 |

FIG. 14 depicts the differential scanning calorimetry (DSC) profile of crystalline Form 3. As shown in FIG. 14, crystalline Form 3 shows a characteristic endotherm with an onset of about 247° C. and a peak of about 249° C. (enthalpy 82.4 J/g).

Crystalline Form 3 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, hydrogen bromide salt, displayed a thermogravimetric analysis (TGA) profile showing a mass loss of about 0.86 wt. % between about 20° C. to about 180° C. (FIG. 15). Crystalline Form 3 was observed to be an anhydrate.

### Example 6

Crystalline Form 4 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, phosphoric acid salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (30 mg) was taken up in MeOH/water (9:1, 14 V) at 50° C. After adding 1 eq. of phosphoric acid, precipitation occurred after cooling to 35° C. The mixture was stirred at 50° C. for 30 min and then cooled to room temperature. The solids were collected by filtration and dried under vacuum. XRPD analysis indicated that the dried material was crystalline with a pattern consistent with Form 4.

Crystalline Form 4 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, phosphoric acid salt, was also prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (30 mg) was taken up in DCM/MeOH (1:1, 22 V) at room temperature. After adding 1 eq. of phosphoric acid, precipitation occurred immediately. The solids were collected by filtration and dried under vacuum. XRPD analysis indicated that the dried material was crystalline with a pattern consistent with Form 4.

The XRPD pattern of crystalline Form 4 is shown in FIG. 16. Characteristic peaks include one or more of the peaks shown in Table 6.

TABLE 6

| Angle | d Value | Peak Height | Rel. Intensity |
|---|---|---|---|
| (deg 2 theta) | (Å) | (counts) | (%) |
| 36.942 | 2.43133 | 97.5517 | 0.1 |
| 35.158 | 2.55050 | 118.399 | 0.2 |
| 34.542 | 2.59452 | 123.69 | 0.2 |
| 34.087 | 2.62811 | 82.3165 | 0.1 |
| 33.470 | 2.67515 | 118.187 | 0.2 |
| 32.683 | 2.73777 | 94.823 | 0.1 |
| 32.078 | 2.78801 | 146.11 | 0.3 |
| 31.391 | 2.84741 | 94.8943 | 0.1 |
| 30.713 | 2.90873 | 136.392 | 0.2 |
| 30.374 | 2.94038 | 117.944 | 0.1 |
| 29.349 | 3.04073 | 237.342 | 0.6 |
| 28.651 | 3.11324 | 130.845 | 0.2 |
| 27.843 | 3.20168 | 216.083 | 0.5 |
| 27.474 | 3.24380 | 130.878 | 0.1 |
| 27.117 | 3.28571 | 180.006 | 0.3 |
| 26.410 | 3.37213 | 178.151 | 0.2 |
| 26.067 | 3.41566 | 517.369 | 1.4 |
| 25.810 | 3.44906 | 310.771 | 0.7 |
| 25.535 | 3.48564 | 364.744 | 0.9 |
| 25.271 | 3.52136 | 240.55 | 0.4 |
| 24.822 | 3.58415 | 146.897 | 0.1 |
| 23.933 | 3.71519 | 219.096 | 0.4 |
| 23.478 | 3.78606 | 410.068 | 1.0 |
| 23.048 | 3.85574 | 256.692 | 0.4 |
| 22.587 | 3.93348 | 190.358 | 0.1 |
| 22.188 | 4.00331 | 1082.53 | 3.3 |
| 21.894 | 4.05635 | 375.156 | 0.8 |
| 21.508 | 4.12821 | 734.628 | 2.1 |
| 20.290 | 4.37333 | 256.382 | 0.5 |
| 19.264 | 4.60379 | 347.409 | 0.8 |
| 18.930 | 4.68435 | 211.268 | 0.3 |
| 18.485 | 4.79587 | 593.22 | 1.7 |
| 17.357 | 5.10492 | 468.999 | 1.3 |
| 16.666 | 5.31520 | 694.934 | 2.1 |
| 14.878 | 5.94965 | 231.849 | 0.4 |
| 14.529 | 6.09170 | 488.056 | 1.3 |
| 13.073 | 6.76656 | 314.005 | 0.7 |
| 12.562 | 7.04081 | 860.763 | 2.6 |
| 11.874 | 7.44713 | 183.26 | 0.2 |
| 11.340 | 7.79691 | 253.167 | 0.4 |
| 10.080 | 8.76850 | 315.555 | 0.6 |
| 9.567 | 9.23729 | 2164.59 | 7.1 |
| 7.882 | 11.20812 | 1473.84 | 4.5 |
| 7.183 | 12.29701 | 387.626 | 0.4 |
| 6.244 | 14.14399 | 2545.51 | 7.6 |
| 4.755 | 18.56898 | 28502.1 | 100.0 |

FIG. 17 depicts the differential scanning calorimetry (DSC) profile of crystalline Form 4. As shown in FIG. 17, crystalline Form 4 shows a characteristic endotherm with an onset of about 172° C. and a peak of about 275° C. (enthalpy 47.2 J/g), a characteristic exotherm with a peak of about 182° C. (enthalpy 36.6 J/g), and a characteristic endotherm with an onset of about 200° C. and a peak of about 204° C. (enthalpy 81.8 J/g).

Crystalline Form 4 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, phosphoric acid salt, displayed a thermogravimetric analysis (TGA) profile showing a mass loss of about 0.26 wt. % between about 20° C. to about 185° C. (FIG. 18). Crystalline Form 4 was observed to be an anhydrate.

### Example 7

Crystalline Form 5-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (32 mg) was taken up in MeOH (10 V) at 50° C. After adding 1.1 eq. of 2M sulfuric acid in ethyl acetate, precipitation occurred after addition of 8 volumes of ethyl acetate at 50° C. The mixture was stirred at 50° C. for 5 min and then cooled to room temperature. The solids were collected by filtration and dried under vacuum. XRPD analysis indicated that the dried material was crystalline with a pattern consistent with Form 5-1.

The XRPD pattern of crystalline Form 5-1 is shown in FIG. 19. Characteristic peaks include one or more of the peaks shown in Table 7.

TABLE 7

| Angle | d Value | Peak Height | Rel. Intensity |
|---|---|---|---|
| (deg 2 theta) | (Å) | (counts) | (%) |
| 39.383 | 2.28605 | 102.476 | 2.4 |
| 38.888 | 2.31400 | 86.4856 | 1.4 |
| 37.168 | 2.41703 | 139.577 | 4.0 |
| 36.557 | 2.45606 | 134.551 | 3.5 |
| 34.900 | 2.56878 | 114.358 | 1.9 |
| 34.431 | 2.60269 | 200.031 | 7.4 |
| 33.432 | 2.67811 | 102.179 | 1.5 |
| 32.119 | 2.78457 | 117.302 | 2.8 |
| 31.303 | 2.85523 | 176.637 | 6.1 |
| 30.561 | 2.92281 | 171.538 | 5.3 |
| 29.786 | 2.99707 | 134.158 | 2.3 |
| 29.511 | 3.02440 | 153.129 | 3.2 |
| 29.008 | 3.07572 | 146.6 | 2.5 |
| 28.406 | 3.13944 | 208.855 | 6.2 |
| 28.114 | 3.17145 | 159.565 | 3.1 |
| 27.806 | 3.20590 | 393.134 | 17.8 |
| 27.509 | 3.23973 | 277.023 | 10.6 |
| 27.251 | 3.26989 | 281.244 | 11.0 |
| 26.580 | 3.35087 | 301.069 | 12.7 |
| 25.444 | 3.49788 | 189.553 | 4.8 |
| 25.033 | 3.55435 | 290.626 | 10.5 |
| 24.476 | 3.63390 | 216.435 | 5.1 |
| 23.486 | 3.78487 | 717.005 | 35.9 |
| 23.091 | 3.84873 | 478.771 | 20.8 |
| 22.185 | 4.00377 | 701.914 | 34.9 |
| 21.753 | 4.08223 | 670.171 | 33.1 |
| 21.193 | 4.18897 | 1725.17 | 100.0 |
| 20.922 | 4.24260 | 748.699 | 38.8 |
| 20.582 | 4.31194 | 375.524 | 15.7 |
| 20.049 | 4.42525 | 272.196 | 10.0 |
| 19.064 | 4.65165 | 150.886 | 3.2 |
| 18.024 | 4.91759 | 477.779 | 23.3 |
| 17.612 | 5.03175 | 138.466 | 1.7 |
| 17.412 | 5.08896 | 170.856 | 3.6 |
| 16.873 | 5.25028 | 867.439 | 47.3 |
| 15.881 | 5.57605 | 445.593 | 20.8 |
| 15.440 | 5.73413 | 351.353 | 15.0 |
| 14.574 | 6.07308 | 300.807 | 12.3 |
| 13.997 | 6.32205 | 175.319 | 4.6 |
| 13.177 | 6.71369 | 186.621 | 5.4 |
| 11.485 | 7.69826 | 195.11 | 5.7 |
| 8.958 | 9.86339 | 430.748 | 18.1 |
| 8.393 | 10.52665 | 896.23 | 46.8 |
| 7.254 | 12.17573 | 713.239 | 34.0 |
| 6.945 | 12.71768 | 378.641 | 12.6 |

FIG. 20 depicts the differential scanning calorimetry (DSC) profile of crystalline Form 5-1. As shown in FIG. 20, crystalline Form 5-1 shows a characteristic endotherm with an onset of about 91° C. and a peak of about 117 CC (enthalpy 58.0 J/g), and a characteristic endotherm with an onset of about 231 CC and a peak of about 236 CC (enthalpy 111.5 J/g).

Crystalline Form 5-1 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt, displayed a thermogravimetric analysis (TGA) profile showing a mass loss of about 4.33 wt. % between about 20° C. to about 130° C. (FIG. 21). Crystalline Form 5-1 was observed to be a methanol solvate (3.7%, ⅔ mol).

### Example 8

Crystalline Form 5-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (32 mg) was taken up in acetone (13 V) at 50° C. After adding 1.1 eq. of 2M sulfuric acid in water, precipitation occurred after 20 min 50° C. The mixture was cooled to room temperature, and the solids were collected by filtration and dried under vacuum. XRPD analysis indicated that the dried material was crystalline with a pattern consistent with Form 5-2.

The XRPD pattern of crystalline Form 5-2 is shown in FIG. 22. Characteristic peaks include one or more of the peaks shown in Table 8.

TABLE 8

| Angle | d Value | Peak Height | Rel. Intensity |
|---|---|---|---|
| (deg 2 theta) | (Å) | (counts) | (%) |
| 38.836 | 2.31697 | 155.453 | 7.0 |
| 38.191 | 2.35462 | 106.394 | 2.2 |
| 38.039 | 2.36369 | 127.825 | 3.9 |
| 36.492 | 2.46025 | 140.025 | 4.2 |
| 35.931 | 2.49737 | 146.01 | 4.5 |
| 35.008 | 2.56108 | 152.384 | 5.3 |
| 32.001 | 2.79449 | 148.541 | 4.6 |
| 31.429 | 2.84404 | 151.201 | 4.9 |
| 31.018 | 2.88082 | 129.543 | 3.3 |
| 29.677 | 3.00790 | 228.604 | 11.0 |
| 28.777 | 3.09987 | 193.268 | 7.1 |
| 28.200 | 3.16197 | 330.784 | 18.6 |
| 27.790 | 3.20770 | 183.574 | 6.0 |
| 26.453 | 3.36663 | 229.24 | 9.3 |
| 25.924 | 3.43419 | 271.918 | 11.9 |
| 25.373 | 3.50754 | 690.401 | 46.6 |
| 24.577 | 3.61923 | 580.118 | 36.4 |
| 24.219 | 3.67193 | 584.131 | 36.6 |
| 23.892 | 3.72138 | 1285.24 | 96.0 |
| 22.813 | 3.89490 | 302.216 | 12.7 |
| 22.131 | 4.01336 | 297.161 | 12.2 |
| 21.862 | 4.06227 | 327.708 | 14.7 |
| 21.574 | 4.11578 | 767.678 | 52.0 |
| 20.796 | 4.26803 | 264.864 | 9.40 |
| 20.257 | 4.38017 | 401.328 | 21.3 |
| 19.909 | 4.45595 | 812.232 | 56.5 |
| 19.178 | 4.62415 | 960.276 | 70.0 |
| 18.679 | 4.74654 | 198.92 | 6.3 |
| 17.951 | 4.93745 | 464.67 | 29.4 |
| 16.138 | 5.48797 | 1292.77 | 100.0 |
| 15.448 | 5.73128 | 399.565 | 24.5 |
| 14.735 | 6.00713 | 138.167 | 2.5 |
| 14.212 | 6.22677 | 196.499 | 7.3 |
| 13.793 | 6.41531 | 311.469 | 17.1 |
| 12.639 | 6.99804 | 596.161 | 41.0 |
| 12.020 | 7.35705 | 224.497 | 9.3 |
| 11.543 | 7.65978 | 310.316 | 16.7 |
| 10.339 | 8.54911 | 176.605 | 4.9 |
| 9.591 | 9.21418 | 683.721 | 47.0 |
| 8.235 | 10.72837 | 561.65 | 35.5 |
| 7.109 | 12.42524 | 360.281 | 15.5 |

FIG. 23 depicts the differential scanning calorimetry (DSC) profile of crystalline Form 5-2. As shown in FIG. 23, crystalline Form 5-2 shows a characteristic endotherm with an onset of about 229° C. and a peak of about 233° C. (enthalpy 100.2 J/g).

Crystalline Form 5-2 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, sulfuric acid salt, displayed a thermogravimetric analysis (TGA) profile showing a mass loss of about 0.3 wt. % between about 20° C. to about 150° C. (FIG. 24). Crystalline Form 5-2 displayed a dynamic vapor sorption (DVS) profile showing a reversable total mass change of about 0.9 wt. % between about 0% to about 80% relative humidity (RH) at 25° C., and a reversable total mass change of about 1.4 wt. % between about 0% to about 90% relative humidity (RH) at 25° C. No form change was observed after DVS analysis. Crystalline Form 5-2 was observed to be an anhydrate.

### Example 9

Crystalline Form 6 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, p-toluenesulfonic acid salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (32 mg) was taken up in MeOH (6 V) at 50° C. After adding 1.1 eq. of 2M p-toluenesulfonic acid in MeOH, precipitation occurred after addition of 25 volumes of ethyl acetate. The mixture was cooled to room temperature, and the solids were collected by filtration and dried under vacuum. XRPD analysis indicated that the dried material was crystalline with a pattern consistent with Form 6.

The XRPD pattern of crystalline Form 6 is shown in FIG. 25. Characteristic peaks include one or more of the peaks shown in Table 9.

TABLE 9

| Angle | d Value | Peak Height | Rel. Intensity |
|---|---|---|---|
| (deg 2 theta) | (Å) | (counts) | (%) |
| 38.836 | 2.31697 | 155.453 | 7.0 |
| 38.191 | 2.35462 | 106.394 | 2.2 |
| 38.039 | 2.36369 | 127.825 | 3.9 |
| 36.492 | 2.46025 | 140.025 | 4.2 |
| 35.931 | 2.49737 | 146.01 | 4.5 |
| 35.008 | 2.56108 | 152.384 | 5.3 |
| 32.001 | 2.79449 | 148.541 | 4.6 |
| 31.429 | 2.84404 | 151.201 | 4.9 |
| 31.018 | 2.88082 | 129.543 | 3.3 |
| 29.677 | 3.00790 | 228.604 | 11.0 |
| 28.777 | 3.09987 | 193.268 | 7.1 |
| 28.200 | 3.16197 | 330.784 | 18.6 |
| 27.790 | 3.20770 | 183.574 | 6.0 |
| 26.453 | 3.36663 | 229.24 | 9.3 |
| 25.924 | 3.43419 | 271.918 | 11.9 |
| 25.373 | 3.50754 | 690.401 | 46.6 |
| 24.577 | 3.61923 | 580.118 | 36.4 |
| 24.219 | 3.67193 | 584.131 | 36.6 |
| 23.892 | 3.72138 | 1285.24 | 96.0 |
| 22.813 | 3.89490 | 302.216 | 12.7 |
| 22.131 | 4.01336 | 297.161 | 12.2 |
| 21.862 | 4.06227 | 327.708 | 14.7 |
| 21.574 | 4.11578 | 767.678 | 52.0 |
| 20.796 | 4.26803 | 264.864 | 9.40 |
| 20.257 | 4.38017 | 401.328 | 21.3 |
| 19.909 | 4.45595 | 812.232 | 56.5 |
| 19.178 | 4.62415 | 960.276 | 70.0 |
| 18.679 | 4.74654 | 198.92 | 6.3 |
| 17.951 | 4.93745 | 464.67 | 29.4 |
| 16.138 | 5.48797 | 1292.77 | 100.0 |
| 15.448 | 5.73128 | 399.565 | 24.5 |
| 14.735 | 6.00713 | 138.167 | 2.5 |
| 14.212 | 6.22677 | 196.499 | 7.3 |
| 13.793 | 6.41531 | 311.469 | 17.1 |
| 12.639 | 6.99804 | 596.161 | 41.0 |
| 12.020 | 7.35705 | 224.497 | 9.3 |
| 11.543 | 7.65978 | 310.316 | 16.7 |
| 10.339 | 8.54911 | 176.605 | 4.9 |
| 9.591 | 9.21418 | 683.721 | 47.0 |
| 8.235 | 10.72837 | 561.65 | 35.5 |
| 7.109 | 12.42524 | 360.281 | 15.5 |

FIG. 26 depicts the differential scanning calorimetry (DSC) profile of crystalline Form 6. As shown in FIG. 26, crystalline Form 6 shows a characteristic endotherm with an onset of about 215° C. and a peak of about 217° C. (enthalpy 79.5 J/g).

Crystalline Form 6 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, p-toluenesulfonic acid salt, displayed a thermogravimetric analysis (TGA) profile showing a mass loss of about 0.6 wt. % between about 20° C. to about 200° C. (FIG. 27). Crystalline Form 6 displayed a dynamic vapor sorption (DVS) profile showing a reversable total mass change of about 0.6 wt. % between about 0% to about 80% relative humidity (RH) at 25° C., and a reversable total mass change of about 0.7 wt. % between about 0% to about 90% relative humidity (RH) at 25° C. No form change was observed after DVS analysis. Crystalline Form 6 was observed to be an anhydrate.

### Example 10

Crystalline Form 7 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, bis-hydrochloride salt, was prepared as followed. N-(4-((2-Methoxy-3-(1-(methyl-d₃)-1H-1,2,4-triazol-3-yl)phenyl)amino)-5-(propanoyl-3,3,3-d₃)pyridin-2-yl) cyclopropanecarboxamide (20.0 mg) was added to isopropyl acetate (0.25 mL) to form a suspension. In a separate vial, a solution was prepared by diluting 0.0094 mL of aqueous HCl (12M) in 0.25 mL of isopropyl acetate. The diluted HCl solution was added dropwise to the suspension with stirring. The resulting slurry was stirred at room temperature for 3 days, after which the solids collected by filtration and air dried at room temperature for 1 hour. XRPD analysis indicated that the dried solid material was crystalline with a pattern consistent with Form 7.

The XRPD pattern of crystalline Form 7 is shown in FIG. 28. Characteristic peaks include one or more of the peaks shown in Table 10.

TABLE 10

| Pos. [°2θ] | Height [cts] | d-spacing [Å] | Rel. Int. [%] |
|---|---|---|---|
| 5.4359 | 904.77 | 16.26 | 25.56 |
| 6.1597 | 1011.08 | 14.35 | 28.56 |
| 9.4377 | 3539.62 | 9.37 | 100.00 |
| 10.2566 | 498.27 | 8.62 | 14.08 |
| 10.8388 | 441.32 | 8.16 | 12.47 |
| 12.3002 | 157.41 | 7.20 | 4.45 |
| 12.8267 | 216.05 | 6.90 | 6.10 |
| 13.0924 | 200.96 | 6.76 | 5.68 |
| 13.7321 | 299.54 | 6.45 | 8.46 |
| 15.2101 | 322.00 | 5.83 | 9.10 |
| 16.0338 | 458.97 | 5.53 | 12.97 |
| 18.4866 | 425.08 | 4.80 | 12.01 |
| 19.0192 | 252.83 | 4.67 | 7.14 |
| 20.1402 | 242.60 | 4.41 | 6.85 |
| 20.4324 | 193.40 | 4.35 | 5.46 |
| 21.3113 | 156.77 | 4.17 | 4.43 |
| 21.6654 | 216.58 | 4.10 | 6.12 |
| 22.0699 | 229.45 | 4.03 | 6.48 |
| 22.7992 | 400.02 | 3.90 | 11.30 |
| 24.2827 | 756.33 | 3.67 | 21.37 |
| 24.7288 | 657.59 | 3.60 | 18.58 |
| 25.1427 | 346.12 | 3.54 | 9.78 |
| 25.9152 | 386.12 | 3.44 | 10.91 |
| 26.2171 | 725.85 | 3.40 | 20.51 |
| 26.7534 | 778.76 | 3.33 | 22.00 |
| 27.6349 | 344.13 | 3.23 | 9.72 |
| 28.1845 | 209.05 | 3.17 | 5.91 |
| 28.7180 | 192.15 | 3.11 | 5.43 |
| 29.1808 | 101.81 | 3.06 | 2.88 |
| 30.0487 | 122.36 | 2.97 | 3.46 |
| 31.0476 | 524.95 | 2.88 | 14.83 |
| 32.1015 | 187.82 | 2.79 | 5.31 |
| 32.8796 | 88.91 | 2.72 | 2.51 |
| 34.0796 | 210.20 | 2.63 | 5.94 |
| 34.7717 | 102.61 | 2.58 | 2.90 |
| 36.8337 | 70.01 | 2.44 | 1.98 |
| 37.5650 | 59.71 | 2.39 | 1.69 |
| 38.5598 | 106.96 | 2.33 | 3.02 |
| 38.9915 | 114.72 | 2.31 | 3.24 |

FIG. 29 depicts the differential scanning calorimetry (DSC) profile of crystalline Form 7. As shown in FIG. 29, crystalline Form 7 shows a characteristic endotherm with a peak of about 111° C., a characteristic endotherm with a peak of about 179° C., and a characteristic endotherm with a peak of about 188° C.

Crystalline Form 7 of N-(4-((2-methoxy-3-(1-(methyl-d₃)-1H-1,2,4-triazol-3-yl)phenyl)amino)-5-(propanoyl-3,3,3-d₃)pyridin-2-yl) cyclopropanecarboxamide, bis-hydrochloride salt, displayed a thermogravimetric analysis (TGA) profile showing a mass loss of about 8.63 wt. % between about 29° C. to about 80° C. (FIG. 30). IC/HPLC test showed that the stoichiometric ratio of Cl⁻ to freebase was 2.0.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, methanesulfonic acid salt (Form 1-1), characterized by a powder X-ray diffraction pattern having a characteristic peak in degrees 2θ at about 24.0.

2. The crystalline form of claim 1, characterized by a powder X-ray diffraction pattern having characteristic peaks in degrees 2θ at about 12.7, 17.1, and 24.0.

3. The crystalline form of claim 1 or 2, characterized by a powder X-ray diffraction pattern having characteristic peaks in degrees 2θ at about 9.5, 12.7, 17.1, 19.1, 20.5, and 24.0.

4. The crystalline form of any one of claims 1-3, characterized by a powder X-ray diffraction pattern having characteristic peaks in degrees 2θ at about 9.5, 12.7, 15.5, 17.1, 19.1, 20.5, 24.0, 24.3, and 26.9.

5. The crystalline form of any one of claims 1-4, characterized by a powder X-ray diffraction pattern having characteristic peaks in degrees 2θ at about 6.9, 7.8, 9.5, 12.7, 15.5, 17.1, 19.1, 20.5, 21.0, 23.7, 24.0, 24.3, and 26.9.

6. The crystalline form of any one of claims 1-5, characterized by a powder X-ray diffraction pattern having characteristic peaks in degrees 2θ at about 6.9, 7.8, 9.5, 12.7, 15.5, 17.1, 19.1, 19.4, 20.5, 20.8, 21.0, 23.7, 24.0, 24.3, 25.5, and 26.9.

7. The crystalline form of any one of claims 1-6, characterized by a differential scanning calorimetry (DSC) profile having a characteristic endotherm with an onset of about 201° C. and a peak of about 202° C.

8. The crystalline form of any one of claims 1-7, characterized by a thermogravimetric analysis (TGA) profile showing a mass loss of about 0.20 wt. % between about 100° C. to about 201° C.

9. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, methanesulfonic acid salt (Form 1-2), characterized by a powder X-ray diffraction pattern having a characteristic peak in degrees 2θ at about 9.9.

10. The crystalline form of claim 9, characterized by a powder X-ray diffraction pattern having characteristic peaks in degrees 2θ at about 7.5, 9.9, and 20.0.

11. The crystalline form of claim 9 or 10, characterized by a powder X-ray diffraction pattern having characteristic peaks in degrees 2θ at about 7.5, 9.9, 13.7, 18.2, 20.0, and 22.5.

12. The crystalline form of any one of claims 9-11, characterized by a powder X-ray diffraction pattern having characteristic peaks in degrees 2θ at about 7.5, 9.2, 9.9, 13.7, 18.2, 20.0, 23.9, 22.5, and 26.5.

13. The crystalline form of any one of claims 9-12, characterized by a powder X-ray diffraction pattern having characteristic peaks in degrees 2θ at about 7.5, 8.3, 9.2, 9.9, 10.4, 13.7, 18.2, 20.0, 21.1, 23.9, 22.5, and 26.5.

14. The crystalline form of any one of claims 9-13, characterized by a differential scanning calorimetry (DSC) profile having a characteristic endotherm with a peak of about 66° C., a characteristic endotherm with an onset of about 122° C. and a peak of about 132° C., a characteristic endotherm with an onset of about 153° C. and a peak of about 161° C., and a characteristic endotherm with an onset of about 195° C. and a peak of about 195° C.

15. The crystalline form of any one of claims 9-14, characterized by a thermogravimetric analysis (TGA) profile showing a mass loss of about 0.70 wt. % between about 20° C. to about 75° C., and a mass loss of about 1.24 wt. % between about 75° C. to about 160° C.

16. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, methanesulfonic acid salt (Form 1-3), characterized by a powder X-ray diffraction pattern having a characteristic peak in degrees 2θ at about 21.0.

17. The crystalline form of claim 16, characterized by a powder X-ray diffraction pattern having characteristic peaks in degrees 2θ at about 9.0, 18.0, and 21.0.

18. The crystalline form of claim 16 or 17, characterized by a powder X-ray diffraction pattern having characteristic peaks in degrees 2θ at about 7.1, 8.1, 9.0, 18.0, 21.0, and 23.3.

19. The crystalline form of any one of claims 16-18, characterized by a powder X-ray diffraction pattern having characteristic peaks in degrees 2θ at about 7.1, 8.1, 9.0, 10.4, 18.0, 20.6, 21.0, 23.3, and 28.5.

20. The crystalline form of any one of claims 16-19, characterized by a powder X-ray diffraction pattern having characteristic peaks in degrees 2θ at about 7.1, 8.1, 9.0, 10.4, 16.5, 18.0, 20.6, 21.0, 23.3, 24.0, 24.2, and 28.5.

21. The crystalline form of any one of claims 16-20, characterized by a powder X-ray diffraction pattern having characteristic peaks in degrees 2θ at about 7.1, 8.1, 9.0, 10.4, 11.1, 15.6, 16.5, 17.5, 18.0, 20.6, 21.0, 23.3, 24.0, 24.2, and 28.5.

22. The crystalline form of any one of claims 16-21, characterized by a differential scanning calorimetry (DSC) profile having a characteristic endotherm with an onset of about 208° C. and a peak of about 209° C.

23. The crystalline form of any one of claims 16-22, characterized by a thermogravimetric analysis (TGA) profile showing no mass loss (0% wt.) between about 20° C. to about 150° C.

24. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, fumaric acid salt (Form 2), characterized by a powder X-ray diffraction pattern having a characteristic peak in degrees 2θ at about 26.1.

25. The crystalline form of claim 24, characterized by a powder X-ray diffraction pattern having characteristic peaks in degrees 2θ at about 8.0, 23.3, and 26.1.

26. The crystalline form of claim 24 or 25, characterized by a powder X-ray diffraction pattern having characteristic peaks in degrees 2θ at about 8.0, 13.5, 22.3, 23.3, 25.6, and 26.1.

27. The crystalline form of any one of claims 24-26, characterized by a powder X-ray diffraction pattern having characteristic peaks in degrees 2θ at about 4.2, 8.0, 8.3, 13.5, 21.0, 22.3, 23.3, 25.6, and 26.1.

28. The crystalline form of any one of claims 24-27, characterized by a powder X-ray diffraction pattern having characteristic peaks in degrees 2θ at about 4.2, 8.0, 8.3, 10.7, 13.5, 18.5, 21.0, 21.3, 22.3, 23.3, 25.6, and 26.1.

29. The crystalline form of any one of claims 24-28, characterized by a powder X-ray diffraction pattern having characteristic peaks in degrees 2θ at about 4.2, 8.0, 8.3, 10.7, 12.5, 13.5, 18.5, 21.0, 22.0, 21.3, 21.7, 22.3, 23.3, 25.6, and 26.1.

30. The crystalline form of any one of claims 24-29, characterized by a differential scanning calorimetry (DSC) profile having a characteristic endotherm with an onset of about 36° C. and a peak of about 57° C., a characteristic endotherm with an onset of about 196° C. and a peak of about 199° C., a characteristic endotherm with an onset of about 202° C. and a peak of about 205° C., a characteristic endotherm with an onset of about 208° C. and a peak of about 209° C., and a characteristic endotherm with an onset of about 213° C. and a peak of about 215° C.

31. The crystalline form of any one of claims 24-30, characterized by a thermogravimetric analysis (TGA) profile showing a mass loss of about 0.42 wt. % between about 20° C. to about 50° C.

32. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, hydrogen bromide salt (Form 3), characterized by a powder X-ray diffraction pattern having a characteristic peak in degrees 2θ at about 9.7.

33. The crystalline form of claim 32, characterized by a powder X-ray diffraction pattern having characteristic peaks in degrees 2θ at about 9.7, 19.5, and 20.5.

34. The crystalline form of claim 32 or 33, characterized by a powder X-ray diffraction pattern having characteristic peaks in degrees 2θ at about 9.7, 19.5, 20.5, 21.2, 23.3, and 26.4.

35. The crystalline form of any one of claims 32-34, characterized by a powder X-ray diffraction pattern having characteristic peaks in degrees 2θ at about 9.7, 17.4, 19.5, 20.5, 21.2, 23.3, 26.4, 27.8, and 29.4.

36. The crystalline form of any one of claims 32-35, characterized by a powder X-ray diffraction pattern having characteristic peaks in degrees 2θ at about 9.7, 17.1, 17.4, 19.5, 20.5, 20.9, 21.2, 22.9, 23.3, 26.4, 27.8, and 29.4.

37. The crystalline form of any one of claims 32-36, characterized by a powder X-ray diffraction pattern having characteristic peaks in degrees 2θ at about 9.7, 17.1, 17.4, 19.5, 20.5, 20.9, 21.2, 22.9, 23.3, 24.8, 26.4, 27.8, 29.4, 30.8, and 32.2.

38. The crystalline form of any one of claims 32-37, characterized by a differential scanning calorimetry (DSC) profile having a characteristic endotherm with an onset of about 247° C. and a peak of about 249° C.

39. The crystalline form of any one of claims 32-38, characterized by a thermogravimetric analysis (TGA) profile showing a mass loss of about 0.86 wt. % between about 20° C. to about 180° C.

40. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, phosphoric acid salt (Form 4), characterized by a powder X-ray diffraction pattern having a characteristic peak in degrees 2θ at about 4.8.

41. The crystalline form of claim 40, characterized by a powder X-ray diffraction pattern having characteristic peaks in degrees 2θ at about 4.8, 6.2, and 9.6.

42. The crystalline form of claim 40 or 41, characterized by a powder X-ray diffraction pattern having characteristic peaks in degrees 2θ at about 4.8, 6.2, 7.9, 9.6, 12.6, and 22.2.

43. The crystalline form of any one of claims 40-42, characterized by a powder X-ray diffraction pattern having characteristic peaks in degrees 2θ at about 4.8, 6.2, 7.9, 9.6, 12.6, 16.7, 18.5, 21.5, and 22.2.

44. The crystalline form of any one of claims 40-43, characterized by a differential scanning calorimetry (DSC) profile having a characteristic endotherm with an onset of about 172° C. and a peak of about 275° C., a characteristic exotherm with a peak of about 182° C., and a characteristic endotherm with an onset of about 200° C. and a peak of about 204° C.

45. The crystalline form of any one of claims 40-44, characterized by a thermogravimetric analysis (TGA) profile showing a mass loss of about 0.26 wt. % between about 20° C. to about 185° C.

46. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, sulfuric acid salt (Form 5-1), characterized by a powder X-ray diffraction pattern having a characteristic peak in degrees 2θ at about 21.2.

47. The crystalline form of claim 46, characterized by a powder X-ray diffraction pattern having characteristic peaks in degrees 2θ at about 8.4, 16.9, and 21.2.

48. The crystalline form of claim 46 or 47, characterized by a powder X-ray diffraction pattern having characteristic peaks in degrees 2θ at about 8.4, 16.9, 20.9, 21.2, 22.2, and 23.5.

49. The crystalline form of any one of claims 46-48, characterized by a powder X-ray diffraction pattern having characteristic peaks in degrees 2θ at about 7.3, 8.4, 16.9, 18.0, 20.9, 21.2, 21.8, 22.2, and 23.5.

50. The crystalline form of any one of claims 46-49, characterized by a powder X-ray diffraction pattern having characteristic peaks in degrees 2θ at about 7.3, 8.4, 9.0, 15.9, 16.9, 18.0, 20.9, 21.2, 21.8, 22.2, 23.1, and 23.5.

51. The crystalline form of any one of claims 46-50, characterized by a powder X-ray diffraction pattern having characteristic peaks in degrees 2θ at about 7.3, 8.4, 9.0, 15.4, 15.9, 16.9, 18.0, 20.6, 20.9, 21.2, 21.8, 22.2, 23.1, 23.5, and 27.8.

52. The crystalline form of any one of claims 46-51, characterized by a differential scanning calorimetry (DSC) profile having a characteristic endotherm with an onset of about 91° C. and a peak of about 117° C., and a characteristic endotherm with an onset of about 231° C. and a peak of about 236° C.

53. The crystalline form of any one of claims 46-52, characterized by a thermogravimetric analysis (TGA) profile showing a mass loss of about 4.33 wt. % between about 20° C. to about 130° C.

54. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, sulfuric acid salt (Form 5-2), characterized by a powder X-ray diffraction pattern having a characteristic peak in degrees 2θ at about 16.1.

55. The crystalline form of claim 54, characterized by a powder X-ray diffraction pattern having characteristic peaks in degrees 2θ at about 16.1, 19.2, and 23.9.

56. The crystalline form of claim 54 or 55, characterized by a powder X-ray diffraction pattern having characteristic peaks in degrees 2θ at about 9.6, 16.1, 19.2, 19.9, 21.6, and 23.9.

57. The crystalline form of any one of claims 54-56, characterized by a powder X-ray diffraction pattern having characteristic peaks in degrees 2θ at about 9.6, 12.6, 16.1, 19.2, 19.9, 21.6, 23.9, 24.2, and 25.4.

58. The crystalline form of any one of claims 54-57, characterized by a powder X-ray diffraction pattern having characteristic peaks in degrees 2θ at about 8.2, 9.6, 12.6, 16.1, 18.0, 19.2, 19.9, 21.6, 23.9, 24.2, 24.6, and 25.4.

59. The crystalline form of any one of claim 54-58, characterized by a powder X-ray diffraction pattern having characteristic peaks in degrees 2θ at about 8.2, 9.6, 12.6, 15.4, 16.1, 18.0, 19.2, 19.9, 20.3, 21.6, 23.9, 24.2, 24.6, 25.4, and 28.2.

60. The crystalline form of any one of claims 54-59, characterized by a differential scanning calorimetry (DSC) profile having a characteristic endotherm with an onset of about 229° C. and a peak of about 233° C.

61. The crystalline form of any one of claims 54-60, characterized by a thermogravimetric analysis (TGA) profile showing a mass loss of about 0.31 wt. % between about 20° C. to about 150° C.

62. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, p-toluenesulfonic acid salt (Form 6), characterized by a powder X-ray diffraction pattern having a characteristic peak in degrees 2θ at about 9.8.

63. The crystalline form of claim 62, characterized by a powder X-ray diffraction pattern having characteristic peaks in degrees 2θ at about 9.8, 19.8, and 22.7.

64. The crystalline form of claim 62 or 63, characterized by a powder X-ray diffraction pattern having characteristic peaks in degrees 2θ at about 6.6, 7.2, 9.8, 19.8, 20.2, and 22.7.

65. The crystalline form of any one of claims 62-64, characterized by a powder X-ray diffraction pattern having characteristic peaks in degrees 2θ at about 6.6, 7.2, 9.8, 16.1, 19.8, 20.2, 22.7, 24.9, and 25.4.

66. The crystalline form of any one of claims 62-65, characterized by a powder X-ray diffraction pattern having characteristic peaks in degrees 2θ at about 6.6, 7.2, 9.8, 15.1, 15.8, 16.1, 19.5, 19.8, 20.2, 22.7, 24.9, and 25.4.

67. The crystalline form of any one of claim 62-66, characterized by a powder X-ray diffraction pattern having characteristic peaks in degrees 2θ at about 6.6, 7.2, 9.8, 13.3, 15.1, 15.8, 16.1, 19.5, 19.8, 20.2, 21.4, 22.7, 24.9, 25.4, and 26.5.

68. The crystalline form of any one of claims 62-67, characterized by a differential scanning calorimetry (DSC) profile having a characteristic endotherm with an onset of about 215° C. and a peak of about 217° C.

69. The crystalline form of any one of claims 62-68, characterized by a thermogravimetric analysis (TGA) profile showing a mass loss of about 0.6 wt. % between about 20° C. to about 200° C.

70. A crystalline form of N-(4-((2-methoxy-3-(1-(methyl-d₃)-1H-1,2,4-triazol-3-yl)phenyl) amino)-5-(propanoyl-3,3,3-d₃)pyridin-2-yl) cyclopropanecarboxamide, bis-hydrochloride salt (Form 7), characterized by a powder X-ray diffraction pattern having a characteristic peak in degrees 2θ at about 9.4.

71. The crystalline form of claim 70, characterized by a powder X-ray diffraction pattern having characteristic peaks in degrees 2θ at about 5.4, 6.2, and 9.4.

72. The crystalline form of claim 70 or 71, characterized by a powder X-ray diffraction pattern having characteristic peaks in degrees 2θ at about 5.4, 6.2, 9.4, 24.3, 26.2, and 26.7.

73. The crystalline form of any one of claims 70-72, characterized by a powder X-ray diffraction pattern having characteristic peaks in degrees 2θ at about 5.4, 6.2, 9.4, 10.3, 24.3, 24.7, 26.2, 26.7, and 31.0.

74. The crystalline form of any one of claims 70-73, characterized by a powder X-ray diffraction pattern having characteristic peaks in degrees 2θ at about 5.4, 6.2, 9.4, 10.3, 10.8, 16.0, 18.5, 24.3, 24.7, 26.2, 26.7, and 31.0.

75. The crystalline form of any one of claims 70-74, characterized by a differential scanning calorimetry (DSC) profile having a characteristic endotherm with a peak of about 111° C., a characteristic endotherm with a peak of about 179° C., and a characteristic endotherm with a peak of about 188° C.

76. The crystalline form of any one of claims 70-75, characterized by a thermogravimetric analysis (TGA) profile showing a mass loss of about 8.63 wt. % between about 29° C. to about 80° C.

77. The crystalline form of any one of claims 1-76, wherein the powder X-ray diffraction pattern was obtained using Cu Kα radiation.

78. A pharmaceutical composition comprising a crystalline form of any one of claims 1-77, and a pharmaceutically acceptable excipient.

79. A pharmaceutical composition formed from the crystalline form of any one of claims 1-77.

80. The pharmaceutical composition of claim 78 or 79, wherein the composition is formulated for oral administration.

81. A method of inhibiting TYK2 activity in a patient in need thereof, comprising administering to the patient a therapeutically effective amount of a crystalline form of any one of claims 1-77, or the pharmaceutical composition of any one of claims 78-79.

82. A method of treating a TYK2-mediated disorder in a patient in need thereof, comprising administering to the patient a therapeutically effective amount of a crystalline form of any one of claims 1-77, or the pharmaceutical composition of any one of claims 78-79.

83. A method of treating one or more of: Crohn's disease, rheumatoid arthritis, psoriasis, systemic lupus erythematosus, ulcerative colitis, psoriatic arthritis, and systemic sclerosis in a patient in need thereof, comprising administering to the patient an effective amount of a crystalline form of any one of claims 1-77, or the pharmaceutical composition of any one of claims 78-79.
